SEQUENCE LISTING (22 sequences)

SEQ ID NO: 1 - DNA, 1562 bp, Geobacillus thermodenitrificans
  source 1..1562: mol_type = genomic DNA
tttcttttggagagtttgatcctggctcaggacgaacgctggcggcgtgcctaatacatgcaagtcgagcggaccgaacgagagcttgctcttgttcggtcagcggcggacgggtgagtaacacgtgggcaacctgcccgcaagaccgggataactccgggaaaccggagctaataccggataacaccaaagaccgcatggtctttggttgaaaggcggcttcggctgtcacttgcggatgggcccgcggcgcattagctagttggtgaggtaacggctcaccaaggcgacgatgcgtagccggcctgagagggtgaccggccacactgggactgagacacggcccagactcctacgggaggcagcagtagggaatcttccgcaatggacgaaagtctgacggagcgacgccgcgtgagcgaagaaggccttcgggtcgtaaagctctgttgtgagggacgaaggagcgccgtttgaataaggcggcgcggtgacggtacctcacgagaaagccccggctaactacgtgccagcagccgcggtaatacgtagggggcgagcgttgtccggaattattgggcgtaaagcgcgcgcaggcggtcctttaagtctgatgtgaaagcccacggctcaaccgtggagagtcattggaaactgggggacttgagtgcaggagaggagagcggaattccacgtgtagcggtgaaatgcgtagagatgtggaggaacaccagtggcgaaggcggctctctggcctgtaactgacgctgaggcgcgaaagcgtggggagcaaacaggattagataccctggtagtccacgccgtaaacgatgagtgctaagtgttagaggggtcacaccctttagtgctgtagctaacgcgataagcactccgcctggggagtacggccgcaaggctgaaactcaaaggaattgacgggggcccgcacaagcggtggagcatgtggtttaattcgaagcaacgcgaagaaccttaccaggtcttgacatcccctgacaacccaagagattgggcgttccccttcggggggacagggtgacaggtggtgcatggttgtcgtcagctcgtgtcgtgagatgttgggttaagtcccgcaacgagcgcaacccttgcctctagttgccagcattcagttgggcactctagagggactgccggctaaaagtcggaggaaggtggggatgacgtcaaatcatcatgccccttatgacctgggctacacacgtgctacaatgggcggtacaaagggctgcgaacccgcgagggggagcgaatcccaaaaagccgctctcagttcggattgcaggctgcaactcgcctgcatgaagccggaatcgctagtaatcgcggatcagcatgccgcggtgaatacgttcccgggccttgtacacaccgcccgtcacaccacgagagcttgcaacacccgaagtcggtgaggtaacccttacgggagccagccgccgaaggtggggcaagtgattggggtgaagtcgtaacaaggtagccgtaccggaaggtgcggctggatcacctcctttcta

SEQ ID NO: 2 - protein, 1082 aa, Geobacillus thermodenitrificans
MKRIGRIFLCAMLFAYVFFAGKPLLSKAEDNKASAYDINEYANSMQPGWNLGNTFDGFDTGKIVLDETAWGNPRVTKELIDKIADEGFKSIRIPITFDTRLSDGPDYTINPEFLARIERVVNWALEANLKVMINIHHDSWRWIADGMVHDHDNTVAKFKAIWTQLADRFKDYNLDLMFESLNEPQFWGAPEDSQRYLNELNSLFYSIVRHSGGNNDIRPLVLPTLNTGSEPEKLDALYNFITQLNDPYIIATIHYYGFWPFSVNIAGVTNFNEETKNDIIHAFDRVHDKFIKNGIPVVIGEYGLLGFDRGTGTIQQGEKLKYFEFMIHYAQEKNLIHMLWDNGQHFGRTSFKWYDAEFGEMLKASWNGRSATADANFIYLKQGKPVQDVTRILQLNGRKFISLQLNGKDLVAGKDYEINGKSLTIKASLLSKLVSNKIREKAVLTATFDKGANWYFHIFNYDTPSLSDSTGTVSNFTIPIKFNGTHLKTMEAKYVDDGSNAGPQNWTSFKEFGYAFSPDYEHDVVTFPYGNERFFLKMAGKMKRIGRIFLCAMLFAYVFFAGKPLLSKAEDNKASAYDINEYANSMQPGWNLGNTFDGFDTGKIVLDETAWGNPRVTKELIDKIADEGFKSIRIPITFDTRLSDGPDYTINPEFLARIERVVNWALEANLKVMINIHHDSWRWIADGMVHDHDNTVAKFKAIWTQLADRFKDYNLDLMFESLNEPQFWGAPEDSQRYLNELNSLFYSIVRHSGGNNDIRPLVLPTLNTGSEPEKLDALYNFITQLNDPYIIATIHYYGFWPFSVNIAGVTNFNEETKNDIIHAFDRVHDKFIKNGIPVVIGEYGLLGFDRGTGTIQQGEKLKYFEFMIHYAQEKNLIHMLWDNGQHFGRTSFKWYDAEFGEMLKASWNGRSATADANFIYLKQGKPVQDVTRILQLNGRKFISLQLNGKDLVAGKDYEINGKSLTIKASLLSKLVSNKIREKAVLTATFDKGANWYFHIFNYDTPSLSDSTGTVSNFTIPIKFNGTHLKTMEAKYVDDGSNAGPQNWTSFKEFGYAFSPDYEHDVVTFPYGNERFFLKMAGK

SEQ ID NO: 3 - protein, 255 aa, Geobacillus thermodenitrificans
MVIIEKETVAQVPVLHVVKKEKREERLPFILFIHGFTSAKEHNLHFGYLLAEAGYRVILPDALHHGERDSSLSERELQLAFWNIVTRTITEIKKIKEELELRNLIQPDRIGLAGTSMGGIVTFGALAEYPWIKAAVSLMGNPMYEAFFDALIETGKKMGVAIPLSDEQLKREKARLMKYDLSRQPEKLAGRPLLIWHGKCDQVVPYSYTYEFYEQIKPLYQGKEENLKFISDDTAGHKVTREAFLETVKWFTKHV

SEQ ID NO: 4 - DNA, 768 bp, Geobacillus thermodenitrificans
  source 1..768: mol_type = genomic DNA
atggtcatcattgaaaaagaaaccgttgcccaagtgcctgtgcttcatgtcgtaaaaaaagaaaagcgggaagaacgattgccgtttattttgtttattcatggatttacaagtgcaaaagaacataatttgcactttgggtacttgcttgccgaggcgggataccgcgtgattttgccggatgcgctgcatcacggtgaacgagattcgtctttatctgagcgtgagctgcaattggcgttctggaatattgtgacgcgtacgattacggaaattaaaaaaatcaaagaggaattagaacttcgcaatctcattcagccggatcggataggacttgccggtacatcgatgggggggattgtcacatttggcgcacttgccgagtatccatggattaaagcggccgtttcgctgatgggaaatccaatgtatgaagcattttttgacgccttaattgaaacaggcaaaaaaatgggagtggcgattccgctttccgatgaacaactaaagcgggaaaaggcgcgattaatgaaatatgacctttctagacagccggagaaattagcgggccgtccgcttcttatttggcatggaaagtgcgaccaagttgtcccttattcttatacatatgagttttatgaacaaataaaaccgctttatcaaggaaaagaagaaaatttgaagtttatttccgatgatacggcagggcataaagtgacaagggaagcgtttttagaaacggtgaaatggtttacgaaacatgtgtaa

SEQ ID NO: 5 - protein, 253 aa, Geobacillus thermodenitrificans
MPYIVEDGVRLYYEEMGSGTPILFIHPPGMGHIVFRHQQSLSSHFRIIMYDMRGNGKSSPSNRPITIPLLADDICRLLNTLDVKQAIICGYSSGGSIALEFALRYPHKVKKLILIGGFSEVCTPLLRYEFLLGIYAAKIGAISLLANVLAKSHEKNKKGQQEIKQYVRLVNKKDLVNMYEKGLTYSCTERLPLLRMPILLIYGARDYYMHPYEKLFRKNVPHAKIIYIENGRHQIPTKHHCELNGILKTYCIT

SEQ ID NO: 6 - DNA, 762 bp, Geobacillus thermodenitrificans
  source 1..762: mol_type = genomic DNA
atgccatatattgtagaagatggcgttcgtctttactatgaagagatgggaagcggtacaccaattttattcattcatccacctggaatgggtcatatcgttttccgtcatcaacaatctttatcaagccacttccgcatcatcatgtatgatatgcgcggcaatgggaaaagcagtccgtcgaatcgtccgattacgattccgctgctagctgatgatatttgccgcttactcaatacacttgacgtaaagcaagcgattatttgcggctattctagcggtggatccattgcgttagaatttgccttacgctatcctcataaagtaaaaaagctcattttaattggcggattctctgaagtatgtactcctttattgcgttatgaattcttgcttggcatttatgcagcaaaaatcggcgctatttctttacttgccaacgtattagccaaatcgcatgaaaaaaataaaaaaggacaacaagaaataaaacaatatgtgcgattggtcaataaaaaagatttagtaaacatgtacgaaaaagggttaacatattcctgtacggagcgccttccattgttgcgcatgccaatccttcttatttatggcgccagagattactatatgcatccatatgagaagctgttcaggaaaaatgtcccccatgcgaaaatcatctatattgaaaacggacgccatcaaattccaaccaaacatcactgcgaattaaacgggattttgaagacatactgtataacgtaa

SEQ ID NO: 7 - protein, 199 aa, Geobacillus thermodenitrificans
MREVVIVEAVRTPVGKRNGVFRNVHPVHLASTVLNEVVKRAGIEKRLVEDIVMGCVTPIAEQGYNIGRLAALEAGFPIEVPAVQINRMCGSGQQAIHFAAQEIRSGDMDITIAAGVESMTKVPILSDGNEKTIPPSLHEKYEFVHQGISAELIAEKYGLTREQLDAYAYESHQRAIRAQEQGIFDQEIVPVEGLDKEGN

SEQ ID NO: 8 - DNA, 597 bp, Geobacillus thermodenitrificans
  source 1..597: mol_type = genomic DNA
atgagagaagttgtcattgtcgaagccgtgcgcacgccagtcggcaagcgaaacggcgtgttccgcaacgtacatcccgttcatttagcgtcaacggtgctcaatgaagtcgtaaaaagagcgggaatcgaaaaacggcttgtcgaagatattgtgatgggatgtgtcacgcccattgcagagcaaggatacaatattgggcggcttgctgcgctggaggcgggatttccaatcgaagtgccagccgtgcaaatcaatcggatgtgcgggtcagggcagcaggcaattcattttgctgcccaagaaattcgctctggcgatatggatattacgattgctgctggcgtcgaaagcatgacgaaggtgccgattttaagcgatggaaacgaaaaaacgattccgccgtcgctgcatgaaaaatatgaatttgttcatcaaggcatttccgcggaattaatcgccgaaaagtacgggctgacgcgcgaacagctggacgcatatgcatacgaaagtcatcagcgcgcgattcgggcgcaagaacaaggaatatttgatcaagaaatcgtgcctgtggaaggtttggataaagaagggaac

SEQ ID NO: 9 - protein, 235 aa, Geobacillus thermodenitrificans
MEFGLAGKTALVAASSQGLGKAIARALVLEGANVMITSRNEEKLQEVAEELNSLHKGRVAYTRTDVTKADDIRQLVAKTVETFGTIDLLVNNAGGPPAGTFETISDKDWQYAFELNLLSYIRLIREALPYLKKKGGKIVNIASSSIKEPIPGLILSNTFRTGIIGLTKTLATEFAPDNILINTVAPGRIATERVAFLDKVNAEKLGITKEEMEARMRSAIPLGRYGTPEEFANVV

SEQ ID NO: 10 - DNA, 705 bp, Geobacillus thermodenitrificans
  source 1..705: mol_type = genomic DNA
atggagttcggattggcgggaaaaacggcgcttgtcgctgcatcgagccaagggcttggcaaagcgattgcccgagcgcttgtgctggaaggagcaaacgtgatgattacaagccgaaatgaggaaaagctgcaggaagtcgccgaggagcttaacagtttacataagggacgcgttgcttatacgcgcaccgatgtgacgaaagcggatgacatccgccaactggtcgccaaaactgtggagacgtttggaacgatcgatttacttgttaacaacgccggcggccctccggcgggaacgttcgaaacaatcagcgacaaagactggcaatatgcgttcgagctcaatttactgagctatattcggttgattcgcgaagcgttgccttatctaaagaaaaaaggcggtaaaattgtcaatatcgcctcgtcgtccatcaaagagcctattccggggctcatcctgtccaatacgttccgcactgggatcatcgggttgacgaaaacgcttgcaacggagttcgcgcctgataatattttgatcaacacagtcgcacctggacggattgctacggaacgggtagctttcttagacaaggtgaacgctgaaaagctcggcattacgaaagaagaaatggaagcgcgcatgagaagcgccattccgctcggccgttacggaacccctgaggagtttgccaacgttgtc

SEQ ID NO: 11 - DNA, 36 bp, synthetic construct
  source 1..36: mol_type = other DNA
taagcaactagtttcgttgttgtcacatcgtcagcc

SEQ ID NO: 12 - DNA, 39 bp, synthetic construct
  source 1..39: mol_type = other DNA
gggttccgcgtgttatacaaaccccttttccttttcatg

SEQ ID NO: 13 - DNA, 36 bp, synthetic construct
  source 1..36: mol_type = other DNA
ttgtataacacgcggaacccctatttgtttattttc

SEQ ID NO: 14 - DNA, 37 bp, synthetic construct
  source 1..37: mol_type = other DNA
ccgactttgcttaccaatgcttaatcagtgaggcacc

SEQ ID NO: 15 - DNA, 30 bp, synthetic construct
  source 1..30: mol_type = other DNA
gcattggtaagcaaagtcggcagtgagccc

SEQ ID NO: 16 - DNA, 32 bp, synthetic construct
  source 1..32: mol_type = other DNA
cgcctgcagaatcagaagcaatacggcggagg

SEQ ID NO: 17 - DNA, 22 bp, synthetic construct
  source 1..22: mol_type = other DNA
acacatactcgcaaaaccaacg

SEQ ID NO: 18 - DNA, 21 bp, synthetic construct
  source 1..21: mol_type = other DNA
atgaacagttggccgaggtgc

SEQ ID NO: 19 - DNA, 36 bp, synthetic construct; identical to SEQ ID NO: 13
  source 1..36: mol_type = other DNA

SEQ ID NO: 20 - DNA, 37 bp, synthetic construct; identical to SEQ ID NO: 14
  source 1..37: mol_type = other DNA

SEQ ID NO: 21 - DNA, 25 bp, synthetic construct
  source 1..25: mol_type = other DNA
tgaatggaccaataataatgactag

SEQ ID NO: 22 - DNA, 24 bp, synthetic construct
  source 1..24: mol_type = other DNA
tgtcgttctgtccactcttaatcc

(19) United States
(12) Patent Application Publication
(10) Pub. No.: US 2025/0230400 A1
(43) Pub. Date: Jul. 17, 2025

(54) COMPOSITIONS AND METHODS FOR DEGRADING LIGNOCELLULOSIC BIOMASS AND PRODUCING POLYHYDROXYALKANOATES

(71) Applicant: SOUTH DAKOTA BOARD OF REGENTS, Pierre, SD (US)

(72) Inventors: Tanvi Govil, Rapid City, SD (US); David R. Salem, Rapid City, SD (US); Rajesh Sani, Rapid City, SD (US)

(21) Appl. No.: 18/853,294

(22) PCT Filed: Apr. 3, 2023
(86) PCT No.: PCT/US2023/065276

§ 371 Date: Oct. 1, 2024

Related U.S. Application Data
(60) Provisional application No. 63/362,429, filed on Apr. 4, 2022.

(51) Int. Cl. C12N 1/20; C12N 1/22; C12N 13/00; C12N 15/75; C12P 1/04; C12P 7/10

(52) U.S. Cl. CPC C12N 1/205; C12N 1/22; C12N 13/00; C12N 15/75; C12P 1/04; C12P 7/10; C12P 2201/00

(57) ABSTRACT

This disclosure relates to the field of bacterial strains and their ability to degrade lignocellulosic biomass. In a preferred embodiment, the present disclosure is directed to a Geobacillus sp. strain. Notably, we have found that the Geobacillus sp. strain has the capability to simultaneously hydrolyze and ferment lignocellulosic biomass to form polyhydroxyalkanoate (PHA). Most preferably, the hydrolysis and fermentation to form PHA takes place in a single step.

### CROSS-REFERENCE

This application claims priority under 35 U.S.C. § 119 to provisional patent application U.S. Ser. No. 63/362,429, filed Apr. 4, 2022. The provisional patent application is herein incorporated by reference in its entirety, including without limitation, the specification, claims, and abstract, as well as any figures, tables, appendices, or drawings thereof.

### SEQUENCE LISTING XML

The instant application contains a sequence listing, which has been submitted in XML file format by electronic submission and is hereby incorporated by reference in its entirety. Said XML file, created on Mar. 27, 2023, is named P13637WO00.xml and is 26,718 bytes in size.

### TECHNICAL FIELD

The present disclosure relates to the field of bacterial strains and their ability to degrade lignocellulosic biomass. In particular, the present disclosure is directed to a Geobacillus sp. strain with the capability to simultaneously hydrolyze and ferment lignocellulosic biomass to polyhydroxyalkanoate (PHA) in a single step.

### BACKGROUND

Cellulosic and lignocellulosic feedstocks and wastes, such as agricultural residues, wood, forestry wastes, sludge from paper manufacture, and municipal and industrial solid wastes, provide a potentially large renewable feedstock for the production of valuable products such as fuels and other chemicals. Cellulosic and lignocellulosic feedstocks and wastes, composed of carbohydrate polymers and aromatic compounds comprising cellulose, hemicellulose, glucans and lignin are generally treated by a variety of chemical, mechanical and enzymatic means to release primarily hexose and pentose sugars, and phenolic alcohol monomers, which can then be fermented to useful products.

Hydrolysates from numerous lignocellulosic materials rich in pentoses and hexoses (e.g., wheat hydrolysate, rice bran, tequila bagasse, liquified wood, maple wood, wheat straw, etc.) have been used for production of PHA and other biopolymers. One challenge in the use of lignocellulosic materials as sources of biopolymers is their recalcitrance and heterogeneity, typically requiring preprocessing of the lignocellulosic materials. Due to their recalcitrant nature, pretreatment cost ($15-25/GJ), along with the high cost of commercially available lignocellulose-hydrolyzing enzymes, such preprocessing methods are expensive, and they decrease the cost-efficacy of the process.

Consequently, there is a need to develop technologies that reduce dependence on preprocessing and pretreatment of the lignocellulosic materials and expensive hydrolyzing enzymes used for hydrolysis and saccharification of pretreated lignocellulosic materials.

### BRIEF SUMMARY OF THE PREFERRED EMBODIMENTS

Disclosed herein is Geobacillus thermodenitrificans strain cnambio1 (NRRL B-68082). This Geobacillus sp. strain provides various advantages over existing bacterial strains. For example, the strain has the capability to simultaneously hydrolyze and ferment lignocellulosic biomass to form polyhydroxyalkanoate (PHA) in a single step.

A preferred embodiment comprises a method for degrading lignocellulosic biomass comprising adding a composition comprising Geobacillus thermodenitrificans strain cnambio1 (NRRL B-68082), or a mutant thereof, to the biomass to form a mixture; and incubating the mixture under conditions suitable for growth of the strain.

A preferred embodiment comprises a method for producing a polyhydroxyalkanoate (PHA) from biomass comprising adding a composition comprising Geobacillus thermodenitrificans strain cnambio1 (NRRL B-68082), or a mutant thereof, to the biomass to form a mixture; incubating the mixture under conditions suitable for growth of the strain to produce the PHA; and extracting the PHA from the cells of the strain.

A preferred embodiment comprises a composition comprising Geobacillus thermodenitrificans strain cnambio1 (NRRL B-68082), or a mutant thereof.

A preferred embodiment comprises a polyhydroxyalkanoate (PHA) extracted from Geobacillus thermodenitrificans strain cnambio1 (NRRL B-68082) or a mutant thereof.

A preferred embodiment comprises a method for transforming exogenous DNA into Geobacillus cells comprising electroporating a suspension comprising the exogenous DNA and cells with (a) two or more square waveform pulses of about 0.75 kV to about 1.25 kV each, or (b) an exponential decay waveform pulse of at least about 2.5 kV.

While multiple embodiments are disclosed, still other embodiments of the present invention will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

The following figures form part of the specification and are included to further demonstrate certain embodiments or various aspects of the invention. In some instances, embodiments of the invention can be best understood by referring to the accompanying figures in combination with the detailed description presented herein. The description and accompanying figures may highlight a certain specific example, or a certain aspect of the invention. However, one skilled in the art will understand that portions of the example or aspect may be used in combination with other examples or aspects of the invention.

FIG. 1A is an image of untreated/unprocessed corn stover (without any physical, chemical, or enzymatic pretreatment).

FIG. 1B is a TEM image of Geobacillus thermodenitrificans strain cnambio1 after aerobic growth in nitrogen-limited media with corn stover at 60° C. for five days. Scale bar: 1.0 μm.

FIG. 1C shows Geobacillus thermodenitrificans strain cnambio1 produced and extracted transparent mcl-PHA.

FIG. 2A shows an FTIR spectrum of an extracted PHA.

FIG. 2B shows an FTIR spectrum of native PHA inside Geobacillus thermodenitrificans strain cnambio1 cells.

FIG. 3A shows a MALDI-TOF mass spectra in negative ion mode of partial pyrolysis products of PHA extracted from Geobacillus thermodenitrificans strain cnambio1, using unprocessed corn stover (CS) as a substrate and propylene carbonate as extraction solvent, in 9-aminoacridine (9-AA) as a matrix. Here, H=3-hydroxyhexanoate; O=3-hydroxyoctanoate; D=3-hydroxydecanoate; DD=3-hydroxydodecanoate. Peaks labeled with a star belong to the MALDI matrix 9-aminoacridine (9-AA). Relative intensity of [M+Na]+ ions of the partial pyrolysis products in the MALDI mass spectrum in IAA, is shown in the inset on the figure.

FIG. 3B shows DSC (Differential Scanning Calorimetry, endotherm down) and TGA (Thermal Gravimetric Analysis) spectra of purified PHA produced by Geobacillus thermodenitrificans strain cnambio1 grown on corn stover, showing a high thermal degradation temperature.

FIG. 3C shows XRD (X-Ray Diffraction) spectrum of purified PHA produced by Geobacillus thermodenitrificans strain cnambio1 grown on corn stover, showing its amorphous nature.

FIG. 3D shows XRD spectra of the produced PHA under varying annealing temperatures for a time of 1 hour, showing development of crystallinity with temperature in a sample that contained residual salt with nucleation properties.

FIG. 4A shows growth profiles in Geobacillus thermodenitrificans strain cnambio1, at 60° C., pH 6.8 in MBSS (Mineral base salt solution) media with different substrates. Concentrations of substrate used: Unprocessed CS (0.5% w/v), BX (Birchwood Xylan, 0.5% w/v), CMC (Carboxymethyl cellulose, 0.1% w/v), Glucose (2% w/v), KL (0.025% w/v). CDW is Cell Dry Weight (g/L); PHA titers are represented as mg/L. All the experiments were performed in triplicates, with data averaged and presented as the mean standard deviation (SD). Error bars smaller than the symbols are not shown.

FIG. 4B shows PHA production in Geobacillus thermodenitrificans strain cnambio1, at 60° C., pH 6.8 in MBSS (Mineral base salt solution) media with different substrates. Concentrations of substrate used: Unprocessed CS (0.5% w/v), BX (Birchwood Xylan, 0.5% w/v), CMC (Carboxymethyl cellulose, 0.1% w/v), Glucose (2% w/v), KL (0.025% w/v). CDW is Cell Dry Weight (g/L); PHA titers are represented as mg/L. All the experiments were performed in triplicates, with data averaged and presented as the mean standard deviation (SD). Error bars smaller than the symbols are not shown.

FIG. 5A shows SEM (Scanning Electron Microscope) images of corn stover before 10 days of incubation with Geobacillus thermodenitrificans strain cnambio1 at 60° C., pH 7.0, and agitation speed of 150 rpm.

FIG. 5B shows SEM (Scanning Electron Microscope) images of corn stover after 10 days of incubation with Geobacillus thermodenitrificans strain cnambio1 at 60° C., pH 7.0, and agitation speed of 150 rpm.

FIG. 6 shows Fourier-transform infrared spectroscopy (FTIR) of PHA obtained from Geobacillus thermodenitrificans strain cnambio1 using chloroform.

FIG. 7A shows thermogravimetric analysis (TGA) of PHA obtained from Geobacillus thermodenitrificans strain cnambio1 using chloroform.

FIG. 7B shows the derivative thermogravimetry (DTG) curve of PHA obtained from Geobacillus thermodenitrificans strain cnambio1 using chloroform.

FIG. 8 shows X-ray diffraction of PHA obtained from Geobacillus thermodenitrificans strain cnambio1 using chloroform.

FIG. 9 shows scans of ¹H nuclear magnetic resonance (NMR) of PHA extracted from Geobacillus thermodenitrificans strain cnambio1 using sodium hypochlorite and chloroform. Here peak numbers, 3=Singlet peak at 1.6 ppm is assigned to methylene protons (—CH₂) adjacent to the β-carbon in the sidechains; 4=Singlet peak at 13 ppm represents methylene protons (—CH₂) of side chains; 5=methyl group (—CH₃) of side chains. Note: when using sodium hypochlorite and chloroform, the signals generated from the main chains are masked.

FIG. 10A shows MALDI-TOF mass spectra in negative ion mode of partial pyrolysis products of PHA extracted from Geobacillus thermodenitrificans strain cnambio1 using sodium hypochlorite and chloroform in 9-aminoacridine (9-AA) as a matrix. Here, H=3-hydroxyhexanoate; O=3-hydroxyoctanoate; D=3-hydroxydecanoate; DD=3-hydroxydodecanoate. Peaks labeled with a star belong to the MALDI matrix.

FIG. 10B shows MALDI-TOF mass spectra in positive mode of partial pyrolysis products of the PHA extracted from Geobacillus thermodenitrificans strain cnambio1 using sodium hypochlorite and chloroform.

FIG. 11 shows molecular weight distribution using GPC waters 1515-2414 system of the PHA extracted from Geobacillus thermodenitrificans strain cnambio1 using sodium hypochlorite and chloroform.

FIG. 12 shows Fourier-transform infrared spectroscopy (FTIR) of PHA obtained from Geobacillus thermodenitrificans strain cnambio1 using propylene carbonate.

FIG. 13A shows thermogravimetric analysis (TGA) of PHA obtained from Geobacillus thermodenitrificans strain cnambio1 using propylene carbonate.

FIG. 13B shows the derivative thermogravimetry (DTG) curve of PHA obtained from Geobacillus thermodenitrificans strain cnambio1 using propylene carbonate.

FIG. 14 shows X-ray diffraction of PHA obtained from Geobacillus thermodenitrificans strain cnambio1 using propylene carbonate.

FIG. 15 shows scans of ¹H nuclear magnetic resonance (NMR) of PHA extracted from cnambio1 strain using propylene carbonate. Here peak numbers, 1=triplet at 5-3 ppm corresponded to methine group (—CH—) of the β carbon; 2=doublet at 2-5 ppm corresponding to methylene group (—CH₂) at the α carbon; 3=Singlet peak at 1.6 ppm is assigned to methylene protons (—CH₂) adjacent to the f-carbon in the side-chains; 4=Singlet peak at 1.3 ppm represents methylene protons (—CH₂) of side chains; 5=methyl group (—CH₃) of side chains.

FIG. 16 shows molecular weight distribution, using Waters 1515-2414 GPC system, of the PHA extracted from Geobacillus thermodenitrificans strain cnambio1 u-sing propylene carbonate.

FIG. 17A-D shows transformation efficiency of Geobacillus thermodenitrificans strain cnambio1 mutants generated by PG2K transformation. FIG. 17A shows the effect of applied voltage (kV) vs resistance (Ω) with an exponential decay waveform pulse. The numbers indicate transformation efficacy (×10² CFU/μg plasmid) (numbers in parentheses represent actual number of CFUs observed on plate using 100 μL of transformation mixture). FIG. 17B shows the effect of applied voltage (kV) vs Pulse duration with a square waveform pulse. The numbers indicate transformation efficacy (×10² CFU/μg plasmid) (numbers in parentheses represent actual number of CFUs observed on plate using 100 μL of transformation mixture). FIG. 17C shows the effect of initial harvestable OD₆₀₀ nm numbers on transformation efficiency. FIG. 17D shows the effect of initial plasmid concentration on transformation efficiency.

FIG. 18 shows the general scheme of overlap extension polymerase chain reaction to create phaZ gene deletion cassette (phaZ_{del} cassette).

FIG. 19A-C shows a 1% (w/v) agarose gel electrophoresis analysis. FIG. 19A shows 870 bp phaZ flanking upstream (1-4), 960 bp Amp^{R} (5-8), 876 bp phaZ flanking downstream (9-12). FIG. 19B shows 1830 bp double gene stitch (phaZ flanking upstream+Amp^{R}). FIG. 19C shows 2706 bp Triple gene stitch (phaZ flanking upstream+Amp^{R}+phaZ flanking downstream. L represents the Tri-dye 1 kb DNA ladder (NEB).

FIG. 20A-C shows creating a phaZ gene knockout. FIG. 20A is a schematic showing the phaZ_{del} cassette is digested with restriction enzymes and ligated to PG2K plasmid prior to transformation into E. coli DH5alpha, TOP10, INV110, and BL21. FIG. 20B is a gel electrophoresis analysis showing two bands for linearized PG2K plasmid (3.8 Kb) and phaZ_{del} cassette (2.8 Kb) that confirms the presence of phaZ gene deletion cassette in the transformant PG2K plasmid. FIG. 20C is a gel electrophoresis analysis showing the identification of two mutants that are negative for the presence of phaZ gene in the genome.

### DETAILED DESCRIPTION

The present disclosure relates to Geobacillus sp. strains that are capable of producing biopolymers, such as polyhydroxyalkanoate, from unprocessed biomass. Also provided are methods of their use, particularly in the production of polyhydroxyalkanoate.

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1′₂, and 4% This applies regardless of the breadth of the range.

The term “about,” as used herein, refers to variation in the numerical quantity that can occur, for example, through typical measuring techniques and equipment, with respect to any quantifiable variable, including, but not limited to, molecular weight, mass, sequence identity, percent homology, pH, temperature, and time. Further, given solid and liquid handling procedures used in the real world, there is certain inadvertent error and variation that is likely through differences in the manufacture, source, or purity of the ingredients used to make the compositions or carry out the methods and the like. The term “about” also encompasses amounts that differ due to different equilibrium conditions for a composition resulting from a particular initial mixture. The term “about” also encompasses these variations. Whether or not modified by the term “about,” the claims include equivalents to the quantities.

A “biologically pure bacterial culture” refers to a culture of bacteria containing no other bacterial species in quantities sufficient to interfere with the replication of the culture or be detected by normal bacteriological techniques. Stated another way, it is a culture wherein virtually all of the bacterial cells present are of the selected strain.

The term “biomass” refers to any cellulosic or lignocellulosic material and includes materials comprising cellulose, and optionally further comprising hemicellulose, lignin, starch, polysaccharides, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, wastepaper, sugar cane bagasse, sorghum, soy, components obtained from milling of grains, trees, branches, roots, leaves, wood chips, sawdust, shrubs and bushes, vegetables, fruits, flowers and animal manure. In one embodiment, the biomass includes corn stover.

The term “cellulase” means one or more (e.g., several) enzymes that hydrolyze a cellulosic material. Such enzymes include endoglucanase(s), cellobiohydrolase(s), beta-glucosidase(s), or combinations thereof.

The term “cellulosic” refers to a composition comprising cellulose.

The term “lignocellulosic” refers to a composition comprising both lignin and cellulose. Lignocellulosic material may also comprise hemicellulose.

As used herein, the term “recombinant” or refers to an organism, microorganism, cell, nucleic acid molecule, or polypeptide that includes at least one genetic alternation or has been modified by introduction of an exogenous nucleic acid molecule, or refers to a cell that has been altered such that the expression of an endogenous nucleic acid molecule or gene can be controlled, where such alterations or modifications are introduced by genetic engineering. Genetic alterations include, for example, modifications introducing expressible nucleic acid molecules encoding proteins or enzymes, or other nucleic acid molecule additions, deletions, substitutions or other functional disruption of the cell's genetic material. Such modifications include, for example, coding regions and functional fragments thereof for heterologous or homologous polypeptides for the referenced species. Additional modifications include, for example, non-coding regulatory regions in which the modifications alter expression of a gene or operon.

While many microorganisms possess the capability of producing polyhydroxyalkanoate or hydrolyzing lignocellulose, a single thermophilic microorganism possessing both capabilities has not been previously reported. Previously non-thermophilic microorganisms have been found to produce polyhydroxyalkanoate from untreated Tequila bagasse, but the microbes were only utilizing the cellulosic components and did not utilize the lignin components. Geobacillus thermodenitrificans strain cnambio1 demonstrates the unusual capability to utilize unprocessed lignocellulose as the sole carbon and energy source and shows the capability to produce a medium chain length PHA (mcl-PHA).

The Geobacillus thermodenitrificans strain cnambio1 expresses lignocellulolytic enzymes including multicopper oxidase, multiphenol oxidase, endo-xylanase, β-xylosidase, endo-glucanase, α-amylase, and pectinase. Moreover, Geobacillus thermodenitrificans strain cnambio1 expresses class IV PHA synthases which transform carbon-based compounds, including C5 pentoses, C6 hexoses, and aromatics, released from biomass degradation into high-molecular weight mcl α-PHA. There is no previous report or commercial technology available to produce bioplastics of improved structural properties from unprocessed lignocellulosic biomass by thermophiles in a single step.

Geobacillus thermodenitrificans strain cnambio1 degrades unprocessed lignocellulosic biomass, obviating the need for expensive physiochemical pretreatment and expensive hydrolytic enzymes, and produces PHA. Simultaneous pretreatment, hydrolysis, and fermentation of lignocellulosic biomass to PHA can occur in a single step in a consolidated bioprocessing (CBP) configuration, which is distinguished from other less highly integrated configurations in that the CBP does not involve a dedicated process step for cellulase or hemicellulase treatment.

Thus, substantially pure cultures, or biologically pure cultures, of such strains are provided.

A sample of Geobacillus thermodenitrificans strain cnambio1 has been deposited with the Agricultural Research Service Culture Collection located at the National Center for Agricultural Utilization Research, Agricultural Research Service, U.S. Department of Agriculture (NRRL), 1815 North University Street, Peoria, IL 61604, U.S.A., under the Budapest Treaty on Nov. 15, 2021, and has been assigned the following accession number: NRRL B-68082.

The strain has been deposited under conditions that assure that access to the strain will be available during the pendency of this patent application to one determined by the Commissioner of Patents and Trademarks to be entitled thereto under 37 CFR 1.14 and 35 USC 122. The deposit is available as required by foreign patent laws in countries wherein counterparts of the subject application, or its progeny, are filed. However, it should be understood that the availability of the deposits does not constitute a license to practice the subject invention in derogation of patent rights granted by governmental action.

In one embodiment, a mutant strain of the Geobacillus thermodenitrificans strain cnambio1 is provided. The term “mutant” refers to a genetic variant of Geobacillus thermodenitrificans strain cnambio1. In one embodiment, the mutant has one or more or all the identifying (functional) characteristics of Geobacillus thermodenitrificans strain cnambio1. In a particular instance, the mutant produces PHA from lignocellulosic biomass at least as well as the parent Geobacillus thermodenitrificans strain cnambio1. Such mutants may be genetic variants having a genomic sequence that has greater than about 85%, greater than about 90%, greater than about 95%, greater than about 98%, or greater than about 99% sequence identity to Geobacillus thermodenitrificans strain cnambio1.

In certain embodiments, the Geobacillus thermodenitrificans strain cnambio1 strain comprises a DNA sequence exhibiting at least 75% sequence identity, at least 80% sequence identity, at least 90% sequence identity, at least 95% sequence identity, at least 96% sequence identity, at least 97% sequence identity, at least 98% sequence identity, or at least 99% sequence identity to SEQ ID NO: 1.

Compositions comprising Geobacillus thermodenitrificans strain cnambio1, or a mutant thereof, are provided. In certain embodiments, the compositions comprise Geobacillus thermodenitrificans strain cnambio1 and a carrier. A variety of carriers are contemplated including but not limited to a liquid, a solid and a combination of a liquid and a solid carrier. In certain embodiments, the carrier is liquid comprising water. In certain embodiments, the compositions may comprise Geobacillus thermodenitrificans strain cnambio1 and one or more additional microbial strains.

Geobacillus thermodenitrificans strain cnambio1 may be genetically modified according to techniques known in the art, e.g., to introduce a polynucleotide that expresses a functional polypeptide, to delete a portion or all of a gene, or to alter (i.e., increase, decrease) the expression levels of an endogenous gene. In certain embodiments, the strain is genetically modified to recombinantly express an enzyme including, for example, a cellulase enzyme. In certain embodiments, the strain is genetically modified to overexpress one or more genes of the phaCAB operon. In certain embodiments, the strain is genetically modified to knockout or knockdown expression of a Pha depolymerase (PhaZ).

In certain embodiments, methods for producing a polyhydroxyalkanoate (PHA) from biomass are provided. The methods comprise adding a composition comprising Geobacillus thermodenitrificans strain cnambio1, or a mutant thereof, to the biomass to form a mixture; incubating the mixture under conditions suitable for growth of the strain to produce the PHA; and extracting the PHA from the cells of the strain.

Polyhydroxyalkanoates are biological polyesters synthesized by a broad range of natural and genetically engineered bacteria as well as genetically engineered plant crops (Braunegg et al., 1998, J. Biotechnology 65:127-161; Madison and Huisman, 1999, Microbiology and Molecular Biology Reviews, 63:21-53; Poirier, 2002, Progress in Lipid Research 41:131-155). These polymers are biodegradable thermoplastic materials, produced from renewable resources, with the potential for use in a broad range of industrial applications (Williams & Peoples, 1996, CHEMTECH 26:38-44). In general, a PHA is formed by enzymatic polymerization of one or more monomer units inside a living cell. Over 100 different types of monomers have been incorporated into the PHA polymers (Steinbüchel and Valentin, 1995, FEMS Microbiol. Lett. 128:219-228.

PHA from Geobacillus thermodenitrificans strain cnambio1 is an amorphous and transparent medium chain length biopolymer that has unusually high thermal stability. In certain embodiments, the PHA is heat stable at a temperature of at least about 350° C. This is an important advantage, since most PHAs start to degrade at temperatures which are too low to permit standard polymer melt-processing (e.g., single-screw and twin-screw extrusion) without significant polymer degradation and consequent decline of polymer properties, especially crucial mechanical properties. Thermoplastics are in increasing demand across the globe and continue to penetrate markets that have been dominated by traditional materials such as metals and ceramics because they offer a gamut of advantages: Plastic compounds weigh less, cost less, are easy to process and mold at relatively low temperature, can be customized, can be reinforced with high stiffness/strength fibers, can incorporate a wide range of multifunctional additives, and can also offer advantages by way of thermal stability, impact strength, and resistance to scratching and abrasion. However, whereas thermoplastic polymers derived from fossil fuels are resistant to enzymatic degradation and are creating a global plastic waste problem, biopolymers produced from microbes are biodegradable. Microbial biopolymers which can be produced and processed economically are therefore of rapidly growing environmental and commercial interest as a sustainable solution to plastic pollution.

In certain embodiments, methods for treating (i.e., hydrolyzing) lignocellulosic biomass are provided. The methods comprise adding a composition comprising Geobacillus thermodenitrificans strain cnambio1, or a mutant thereof, to the biomass to form a mixture; and incubating the mixture under conditions suitable for growth of the strain. The resulting soluble products can be used by Geobacillus thermodenitrificans strain cnambio1 or by another microorganism to produce PHA or any other economically desirable biopolymer including, for example, exopolysaccharide (EPS) or Bio-rubber.

Any suitable biomass material can be used. In certain embodiments, the biomass comprises wood, wood pulp, wood chips, sawdust, hardwood, softwood, newsprint, cardboard, paper pulp, corn fiber, corn grain, corn cobs, corn husks, corn stover, grasses, wheat, wheat straw, barley, barley straw, oat straw, oat hulls, hay, rice, rice straw, switchgrass, cord grass, rye grass, miscanthus, reed canary grass, waste paper, paper, fruit pulp, vegetable pulp, distillers grain, rice hulls, rice straw, cotton, hemp, flax, sisal, sugar cane bagasse, sugar cane straw, beet pulp, sorghum, soy, soybean stover, canola straw, flowers, or any mixtures thereof. In one embodiment, the biomass comprises corn stover. In certain embodiments, the lignocellulosic biomass is the sole carbon source in the mixture.

Media compositions for the fermentation of biomass material to PHA by Geobacillus thermodenitrificans strain cnambio1 are provided. In certain embodiments, the media compositions comprise α carbon source (e.g., biomass) and a nitrogen source. In certain embodiments, the carbon source and nitrogen source are present at ratio of about 10:1 to about 20:1, about 11:1 to about 19:1, about 12:1 to about 18:1, about 13:1 to about 17:1, about 14:1 to about 16:1, or any value or subrange within the recited ranges, including endpoints. For example, the carbon source and nitrogen source ratio can be about 10:1, about 11:1, about 12:1, about 13:1, about 14:1, about 15:1, about 16:1, about 17:1, about 18:1, about 19:1, or about 20:1.

In certain embodiments, the media composition comprises about 1 g/L to about 10 g/L, about 2 g/L to about 8 g/L, about 3 g/L to about 7 g/L, or about 4 g/L to about 6 g/L of α carbon source (e.g., biomass), or any value or subrange within the recited ranges, including endpoints. For example, the media composition can comprise about 1 g/L, about 2 g/L, about 3 g/L, about 4 g/L, about 5 g/L, about 6 g/L, about 7 g/L, about 8 g/L, about 9 g/L, or about 10 g/L of α carbon source.

In certain embodiments, the media composition comprises about 0.05 g/L to about 1 g/L, about 0.1 g/L to about 0.9 g/L, about 0.15 g/L to about 0.8 g/L, about 0.2 g/L to about 0.7 g/L, or about 0.25 g/L to about 0.6 g/L of a nitrogen source, or any value or subrange within the recited ranges, including endpoints. For example, the media composition can comprise about 0.05 g/L, about 0.1 g/L, about 0.15 g/L, about 0.2 g/L, about 0.25 g/L, about 0.3 g/L, about 0.35 g/L, about 0.4 g/L, about 0.45 g/L, about 0.5 g/L, about 0.55 g/L, about 0.6 g/L, about 0.65 g/L, about 0.7 g/L, about 0.75 g/L, about 0.8 g/L, about 0.85 g/L, about 0.9 g/L, about 0.95 g/L, or about 1 g/L of a nitrogen source. Examples of nitrogen sources include, but are not limited to, inorganic ammonium salts such as ammonium sulfate, ammonium chloride, or ammonium phosphate, inorganic nitrate salts such as potassium nitrate or sodium nitrate, peptone, tryptone, yeast extract, urea, and corn steep liquor.

In certain embodiments, the media composition comprises a phosphorous source. In certain embodiments, the media composition comprises about 1 g/L to about 5 g/L, about 2 g/L to about 4 g/L, or about 1.5 g/L to about 3.5 g/L of a phosphorous source, or any value or subrange within the recited ranges, including endpoints. For example, the media composition can comprise about 1 g/L, about 1.5 g/L, about 2 g/L, about 2.5 g/L, about 3 g/L, about 3.5 g/L, about 4 g/L, about 4.5 g/L or about 5 g/L of a phosphorous source. Examples of phosphorous sources include, but are not limited to, inorganic phosphate salts such as monopotassium phosphate, dipotassium phosphate, disodium phosphate, monosodium phosphate, diammonium phosphate, monoammonium phosphate, or calcium phosphate.

A non-limiting example of a suitable media composition comprises 5 g/L biomass as a carbon source (e.g., corn stover), 0.25 g/L potassium nitrate, 2 g/L monopotassium phosphate, 0.5 g/L magnesium sulphate heptahydrate, 0.1 g/L yeast extract, 0.05 g/L sodium molybdate, and 0.02 g/L calcium chloride dihydrate, and 0.5 g/L sodium bicarbonate.

In certain embodiments, the biomass is pretreated, whereby the biomass has been at least partially separated into cellulose, hemicellulose, and lignin by a mild pretreatment to increase the surface area or accessibility of the material. In certain embodiments, the biomass is unprocessed/untreated, whereby the biomass has not been subjected to any physical, chemical, electrical, or enzymatic treatment.

Any pretreatment process known in the art can be used to disrupt plant cell wall components of the biomass (Chandra et al., 2007, Adv. Biochem. Engin. Biotechnol. 108: 67-93; Galbe and Zacchi, 2007, Adv. Biochem. Engin. Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J Mol. Sci. 9: 1621-1651; Yang and Wyman, 2008, Biofuels Bioproducts and Biorefining-Biofpr 2: 26-40). The biomass can also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and enzymatic pretreatment. Additional pretreatments include ammonia percolation, ultrasound, electroporation, microwave, supercritical CO₂, supercritical H₂O, ozone, ionic liquid, and gamma irradiation pretreatments.

In steam pretreatment, the biomass is heated to disrupt the plant cell wall components, including lignin, hemicellulose, and cellulose to make the cellulose and other fractions, e.g., hemicellulose, accessible to enzymes. The biomass is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the biomass is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

The term “chemical treatment” refers to any chemical pretreatment that promotes the separation and/or release of cellulose, hemicellulose, and/or lignin. Such a pretreatment can convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, Bioresource Technology 855: 1-33; Schell et al., 2004, Bioresource Technology 91: 179-188; Lee et al., 1999, Adv. Biochem. Eng. Biotechnol. 65: 93-115).

Several methods of pretreatment under alkaline conditions can also be used. These alkaline pretreatments include, but are not limited to, sodium hydroxide, lime, wet oxidation, ammonia percolation (APR), and ammonia fiber/freeze expansion (AFEX) pretreatment.

Lime pretreatment is performed with calcium oxide or calcium hydroxide at temperatures of 85-150° C. and residence times from 1 hour to several days (Wyman et al., 2005, Bioresource Technology 96: 1959-1966; Mosier et al., 2005, Bioresource Technology 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber expansion (AFEX) involves treating the cellulosic material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, Bioresource Technology 96: 673-686, and U.S. Published Application 2002/0164730.

The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The biomass can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. The physical and chemical pretreatments can be carried out sequentially or simultaneously, as desired.

The present methods can be conducted at any suitable pH and temperature. In some embodiments, the incubation is carried out at a pH and temperature that is at or near the optimum for the growth of Geobacillus thermodenitrificans strain cnambio1. For example, in some embodiments, the incubation is carried out at about 30° C. to about 80° C., or any suitable temperature therebetween, for example a temperature of about 30° C., about 35° C., about 40° C., about 45° C., about 50° C., about 55° C., about 60° C., about 65° C., about 70° C., about 75° C., about 80° C., or any temperature therebetween, and a pH of about 6.0 to about 9.0, or any pH therebetween (e.g., about 6.0, about 6.5, about 7.0, about 7.5, about 8.0, about 8.5, about 9.0, or any suitable pH therebetween). In certain embodiments, the incubating is at a temperature of about 55° C. to about 60° C. In certain embodiments, the incubating is at a pH of about 7 to about 8.

The incubation can be carried out for a time period between about 12 hours and about 30 days, preferably between about 24 hours and about 15 days, more preferably between about 5 days and about 10 days, or any suitable time therebetween, for example a time of about 12 hours, about 24 hours, about 36 hours, about 2 days, about 3 days, about 4 days, about 5 days, about 6 days, about 7 days, about 8 days, about 9 days, about 10 days, about 15 days, about 20 days, about 25 days, about 30 days, or for any time therebetween. In certain embodiments, the incubating is carried out for a time of about 24 hours to about 10 days.

Embodiments of the method include the step of extracting the PHA from the cells of Geobacillus thermodenitrificans strain cnambio1. The PHA can be extracted according to methods known in the art, including but not limited to, fractionation, dialysis, affinity isolation, sequential surfactant, and hypochlorite treatment, and other mechanical or chemical isolation and/or purification techniques. In certain embodiments, extraction is with an organic solvent such as chloroform, propylene carbonate, dichloromethane, acetone, or ethyl acetate. PHA can be separated from the cell components or other undesired substances by solvent extraction and aqueous digestion. Centrifugation, lyophilization, and chemical digestion with chemicals such as sodium hydroxide, chloroform, and methylene chloride can also be used. Fluid extraction using gases such as carbon dioxide can be used. Sonication, freeze-drying, homogenization, and enzymatic digestion can be used to disrupt cells and liberate PHA. Other methods of dissolution and precipitation can also be used.

Genetic engineering of thermophiles has been generally considered a barrier obstructing the convenient genetic manipulation of these organisms. These barriers include the complexity of bacterial transformation, often due to a tough cell membrane that is weakly penetrable (many thermophiles form endospores), or the lack of well-established genetic toolkits. Geobacillus sp. strain cnambio1 is a Gram-positive non-model thermophilic host belonging to the genus Geobacillus, for which the number of available promoters and plasmids is relatively limited, and genetic engineering tools are still developing. Strain level variation in restriction-modification systems is one big critical barrier that hinders the applicability of any gene editing approach universally amongst the member species and strains. Indeed, effective engineering of Geobacillus spp. is a challenge that needs a reliable and convenient transformation method that is strain specific.

Methods for transforming exogenous DNA into Geobacillus cells are provided. Electroporation is used in the present disclosure to facilitate the introduction of DNA into Geobacillus cells. Electroporation is the process of using a pulsed electric field to transiently permeabilize cell membranes, allowing macromolecules, such as DNA, to pass into cells.

In certain embodiments, the methods comprise electroporating a suspension comprising exogenous DNA and Geobacillus cells with one or more square waveform pulses. In certain embodiments, two or more (e.g., 2, 3, 4, 5, or 6) square waveform pulses can be applied. In certain embodiments, the electroporating comprises four square waveform pulses.

In certain embodiments, a square waveform pulse of about 0.5 kilovolts (kV) to about 1.5 kV each is applied. In certain embodiments, a square waveform pulse of about 0.75 kV to about 1.25 kV each is applied. In certain embodiments, a square waveform pulse of about 0.5 kV to about 1.0 kV, about 0.5 kV to about 0.75 kV, about 0.75 kV to about 1.0 kV, about 1.0 kV to about 1.25 kV, or about 1.25 kV to about 1.5 kV each is applied. In certain embodiments, a square waveform pulse of about 1.0 kV each is applied. The square waveform pulse voltage may be any value or subrange within the recited ranges, including endpoints.

In certain embodiments, each square waveform pulse has a duration of about 2.5 ms to about 7.5 ms, about 3 ms to about 7 ms, about 3.5 ms to about 6.5 ms, about 4 ms to about 6 ms, or any value or subrange within the recited ranges, including endpoints. For example, the square waveform pulse duration can be about 2.5 ms, about 3 ms, about 3.5 ms, about 4 ms, about 4.5 ms, about 5 ms, about 5.5 ms, about 6 ms, about 6.5 ms, or about 7.5 ms.

In certain embodiments, the methods comprise electroporating a suspension comprising exogenous DNA and Geobacillus cells with an exponential decay waveform pulse. In certain embodiments, an exponential decay waveform pulse of at least about 2.5 kV is applied. In certain embodiments, an exponential decay waveform pulse of about 2.5 kV to about 3.5 kV is applied. In certain embodiments, an exponential decay waveform pulse of about 2.5 kV to about 3.0, about 2.5 kV to about 2.75 kV, about 2.75 kV to about 3.0 kV, about 3.0 kV to about 3.25 kV, or about 3.25 kV to about 3.5 kV is applied. The exponential decay waveform pulse voltage may be any value or subrange within the recited ranges, including endpoints.

In certain embodiments, the exponential decay waveform pulse has a resistance of about 400Ω to about 800Ω, about 450Ω to about 750Ω, about 500Ω to about 700Ω, or any value or subrange within the recited ranges, including endpoints. For example, the exponential decay waveform pulse can have a resistance of about 400Ω, about 450Ω, about 500Ω, about 550Ω, about 600Ω, about 650Ω, about 700Ω, about 750Ω, or about 800Ω.

In certain embodiments, the exponential decay waveform pulse has a capacitance of about 10 microfarads (μF) to about 50 μF, about 15 μF to about 45 μF, about 20 μF to about 40 μF, or any value or subrange within the recited ranges, including endpoints. For example, the exponential decay waveform pulse can have a capacitance of about 10 μF, about 15 μF, about 20 μF, about 25 μF, about 30 μF, about 35 μF, about 40 NF, about 45 NF, or about 50 NF.

In certain embodiments, the cells are grown to an optical density at 600 nm (OD₆₀₀) of about 1.4 to about 1.8 prior to the electroporation. In certain embodiments, the cells are grown to an OD₆₀₀ of about 1.4 to about 1.6, about 1.4 to about 1.5, about 1.5 to about 1.6, about 1.6 to about 1.8, about 1.6 to about 1.7, or about 1.7 to about 1.8. The OD₆₀₀ value to which the cells are grown may be any value or subrange within the recited ranges, including endpoints.

In certain embodiments, the suspension comprises about 800 nanograms (ng) to about 1200 ng of exogenous DNA. In certain embodiments, the suspension comprises about 800 ng to about 1100 ng, about 800 ng to about 1000 ng, about 800 ng to about 900 ng, about 900 ng to about 1200 ng, about 1000 ng to about 1200 ng, or about 1100 ng to about 1200 ng. The amount of exogenous DNA in the suspension may be any value or subrange within the recited ranges, including endpoints. For example, the suspension can comprise about 800 ng, about 850 ng, about 900 ng, about 950 ng, about 1000 ng, about 1050 ng, about 1100 ng, about 1150 ng, or about 1200 ng of exogenous DNA. The exogenous DNA can be linear DNA or circular DNA.

Applicants have found that optimal transformation efficiencies were achieved by growing cells to an OD₆₀₀ of 1.5-1.6, mixing the cells with approximately 1000 ng of exogenous DNA (e.g., plasmid), and applying either a single exponential decaying electroporation pulse at 3 kV, 600Ω, and 25 μF, or four consecutive square wave electroporation pulses of 1 kV, 5 ms duration.

The following numbered embodiments also form part of the present disclosure:

1. A method for degrading lignocellulosic biomass, the method comprising: adding a composition comprising Geobacillus thermodenitrificans strain cnambio1 (NRRL B-68082), or a mutant thereof, to the biomass to form a mixture; and incubating the mixture under conditions suitable for growth of the strain.

2. The method of embodiment 1, wherein the biomass is unprocessed.

3. The method of embodiment 1, wherein the biomass is pretreated.

4. The method of any one of embodiments 1-3, wherein the lignocellulosic biomass is the sole carbon and energy source in the mixture.

5. The method of any one of embodiments 1-4, wherein the biomass comprises corn stover.

6. A method for producing a polyhydroxyalkanoate (PHA) from biomass, the method comprising: adding a composition comprising Geobacillus thermodenitrificans strain cnambio1 (NRRL B-68082), or a mutant thereof, to the biomass to form a mixture; incubating the mixture under conditions suitable for growth of the strain to produce the PHA; and extracting the PHA from the cells of the strain.

7. The method of embodiment 6, wherein the biomass is unprocessed.

8. The method of embodiment 6, wherein the biomass is pretreated.

9. The method of any one of embodiments 1-8, wherein the biomass comprises wood, wood pulp, wood chips, sawdust, hardwood, softwood, newsprint, cardboard, paper pulp, corn fiber, corn grain, corn cobs, corn husks, corn stover, grasses, wheat, wheat straw, barley, barley straw, oat straw, oat hulls, hay, rice, rice straw, switchgrass, cord grass, rye grass, miscanthus, reed canary grass, waste paper, paper, fruit pulp, vegetable pulp, distillers grain, rice hulls, rice straw, cotton, hemp, flax, sisal, sugar cane bagasse, sugar cane straw, beet pulp, sorghum, soy, soybean stover, canola straw, flowers, or any mixtures thereof.

10. The method of any one of embodiments 6-9, wherein the PHA is a medium chain length PHA (mcl-PHA).

11. The method of any one of embodiments 6-10, wherein the PHA has a number average molecular weight between about 10,000 and about 15,000 g/mol and a weight average molecular weight between about 20,000 and about 30,000 g/mol.

12. The method of any one of embodiments 6-11, wherein the PHA is heat stable at a temperature of at least about 350° C.

13. The method of any one of embodiments 6-12, wherein the extracting is with an organic solvent.

14. The method of embodiment 13, wherein the organic solvent comprises chloroform or propylene carbonate.

15. The method of any one of embodiments 1-14, wherein the incubating is at a temperature of about 55° C. to about 60° C.

16. The method of any one of embodiments 1-15, wherein the incubating is at a pH of about 7 to about 8.

17. The method of any one of embodiments 1-16, wherein the incubating is carried out for a time of about 24 hours to about 10 days.

18. The method of any one of embodiments 1-17, wherein the composition comprises Geobacillus thermodenitrificans strain cnambio1 (NRRL B-68082).

19. The method of any one of embodiments 1-18, wherein the strain further comprises at least one genetic modification.

20. The method of embodiment 19, wherein the strain is genetically modified to: (a) recombinantly express an enzyme, optionally wherein the enzyme is a cellulase; (b) overexpress one or more genes of the phaCAB operon; or (c) knockout or knockdown expression of a Pha depolymerase (PhaZ).

21. A composition comprising Geobacillus thermodenitrificans strain cnambio1 (NRRL B-68082), or a mutant thereof.

22. The composition of embodiment 21, wherein the strain comprises a 16S ribosomal RNA sequence having at least about 95%, at least about 98%, or at least about 99% sequence identity with the sequence of SEQ ID NO: 1.

23. The composition of embodiment 21 or embodiment 22, further comprising a carrier.

24. The composition of any one of embodiments 21-23, wherein the strain further comprises at least one genetic modification.

25. The composition of embodiment 24, wherein the strain is genetically modified to: (a) recombinantly express an enzyme, optionally wherein the enzyme is a cellulase; (b) overexpress one or more genes of the phaCAB operon; or (c) knockout or knockdown expression of a Pha depolymerase (PhaZ).

26. A polyhydroxyalkanoate (PHA) extracted from Geobacillus thermodenitrificans strain cnambio1 (NRRL B-68082) or a mutant thereof.

27. The PHA of embodiment 26, wherein the PHA is a medium chain length PHA (mcl-PHA).

28. The PHA of embodiment 26 or embodiment 27, wherein the PHA has a number average molecular weight between about 10,000 and about 15,000 g/mol and a weight average molecular weight between about 20,000 and about 30,000 g/mol.

29. The PHA of any one of embodiments 26-28, wherein the PHA is heat stable at a temperature of at least about 350° C.

30. A method for transforming exogenous DNA into Geobacillus cells, the method comprising: electroporating a suspension comprising the exogenous DNA and cells with (a) two or more square waveform pulses of about 0.75 kV to about 1.25 kV each, or (b) an exponential decay waveform pulse of at least about 2.5 kV.

31. The method of embodiment 30, wherein the electroporating comprises four square waveform pulses.

32. The method of embodiment 30 or embodiment 31, wherein each square waveform pulse has a duration of about 5 ms.

33. The method of embodiment 30, wherein the exponential decay waveform pulse has resistance of about 400Ω to about 800Ω.

34. The method of embodiment 30 or embodiment 33, wherein the exponential decay waveform pulse has a capacitance of about 10 μF to about 50 μF.

35. The method of any one of embodiments 30-34, wherein the cells are grown to an OD₆₀₀ of about 1.4 to about 1.8.

36. The method of any one of embodiments 30-35, wherein the suspension comprises about 800 ng to about 1200 ng of the exogenous DNA.

37. The method of any one of embodiments 30-36, wherein the exogenous DNA is linear or circular.

38. The method of any one of embodiments 30-37, wherein the cells are Geobacillus thermodenitrificans strain cnambio1 (NRRL B-68082) cells.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

One or more preferred embodiments are shown in the following non-limiting Examples. It should be understood that these Examples, while indicating certain embodiments of the inventions, are given by way of illustration only. From the above discussion and these Examples, one skilled in the art can ascertain the essential characteristics of the disclosed inventions, and without departing from the spirit and scope thereof, can make various changes and modifications of the embodiments of the inventions to adapt to various usages and conditions. Thus, various modifications of the embodiments of the inventions, in addition to those shown and described herein, will be apparent to those skilled in the art from the foregoing description. Such modifications are also intended to fall within the scope of the appended claims.

### Example 1: Characterization of Geobacillus thermodenitrificans Strain Cnambio1

In 2017, a thermophilic bacterium (cnambio1) was isolated from soil samples collected from the Rapid City Solid Waste Division, SD (USA) and identified as Geobacillus thermodenitrificans on the basis of phenotypic features and genotypic investigations.

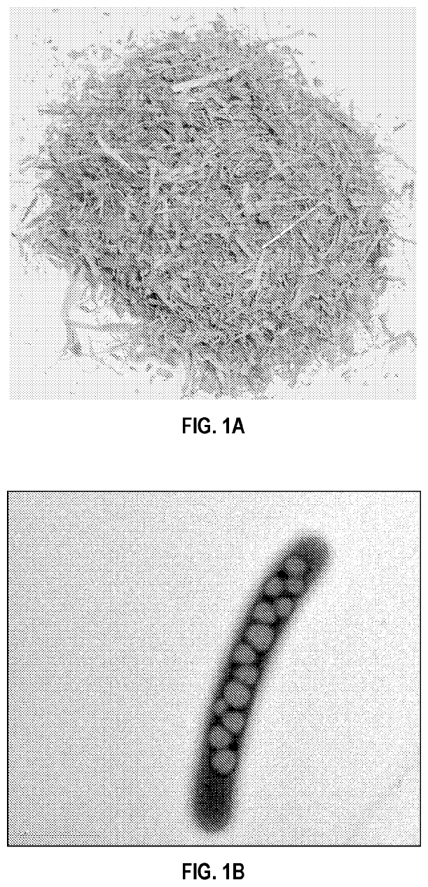

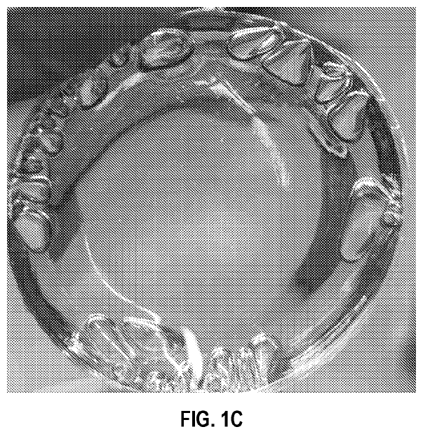

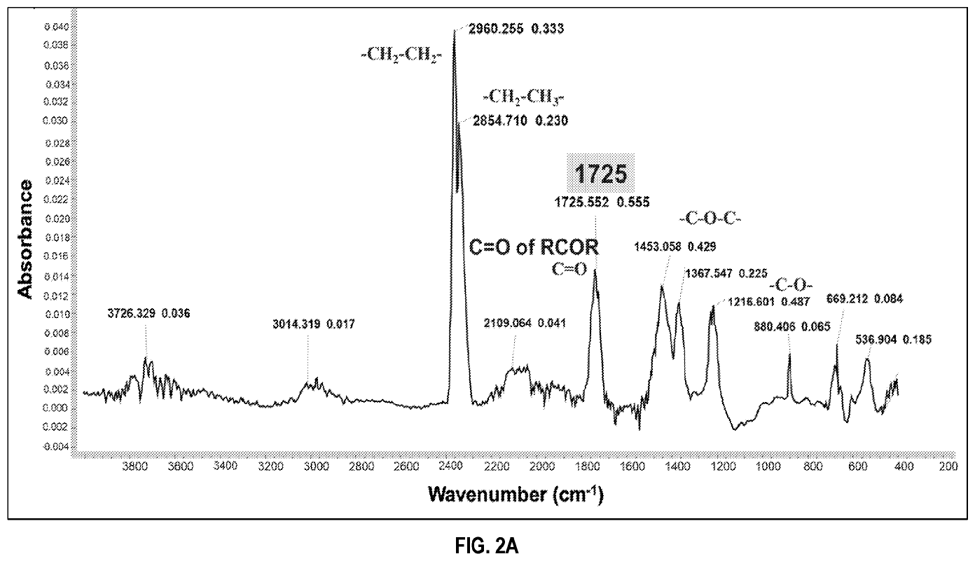

Optimum growth of Geobacillus thermodenitrificans strain cnambio1 occurs between 55° C. and 60° C. and at a pH between 7 and 8. This bacterium has the genomic and metabolic machinery to accumulate PHA which was detected by transmission electron microscopy (TEM) imaging (FIG. 1B). Table 1 shows in silico characterization of the Geobacillus thermodenitrificans strain cnambio1 whole genome using BLAST algorithm+KEGG+UniProt.

TABLE 1

| Geobacillus | | | | |
|---|---|---|---|---|
| thermodenitrificans | | Protein | | |
| strain cnambio1genes | Closest Hit | identity | Role | Reaction sequence |
| Acetoacetyl-CoA | Long chain | fadD | Fatty acid | a long-chain fatty |
| synthetase | acyl-CoA | | degradation | acid + ATP + CoA = a long- |
| (EC 6.2.1.16) | synthetase | | (Beta- | chain fatty acyl- |
| | | | oxidation). | CoA + AMP + diphosphate |
| 3-ketoacyl-CoA thiolase | β-ketoadipyl | fadA | Fatty acid | a 2,3,4-saturated fatty acyl |
| (EC 2.3.1.16) | CoA thiolase | | biosynthesis | CoA + acetyl-CoA ← a 3- |
| | | | | oxoacyl-CoA + coenzyme A |
| Acetyl-CoA | acetyl-CoA | PhaA | PHA | 2 acetyl-CoA ↔ acetoacetyl- |
| acetyltransferase | acetyltransferase | | biosynthesis | CoA + coenzyme A |
| (EC 2.3.1.9) | | | | |
| Enoyl-CoA hydratase | enoyl-CoA | CaiD/fadB | β oxidation | a (3S)-3-hydroxyacyl-CoA + |
| (EC 4.2.1.17) | hydratase/(S)- | | deviation/PHA | NAD⁺ → a 3-oxoacyl-CoA + |
| | 3- | | biosynthesis | NADH + H⁺ |
| | hydroxyacyl- | | | |
| | CoA | | | |
| | dehydrogenase | | | |
| 3-hydroxybutyryl-CoA | 3- | PhaR | PHA | acetoacetyl-CoA + NADPH + |
| dehydrogenase | hydroxybutyryl- | | biosynthesis | H+ → (S)-3- |
| (EC 1.1.1.157) | CoA | | | hydroxybutanoyl-CoA + |
| | dehydrogenase | | | NADP+ |
| D-β-hydroxybutyrate | D-β- | bhbP | Gluconate | |
| permease | hydroxybutyrate | | transporter | |
| | permease | | | |

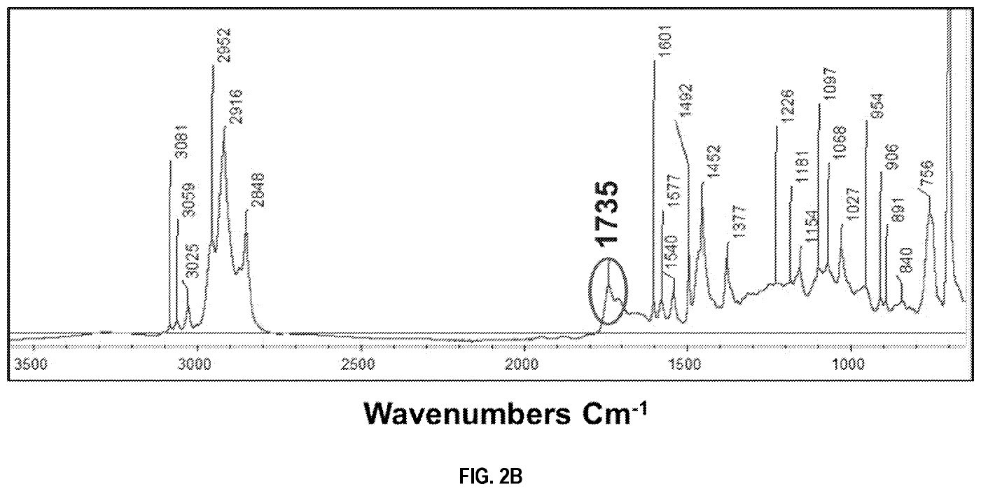

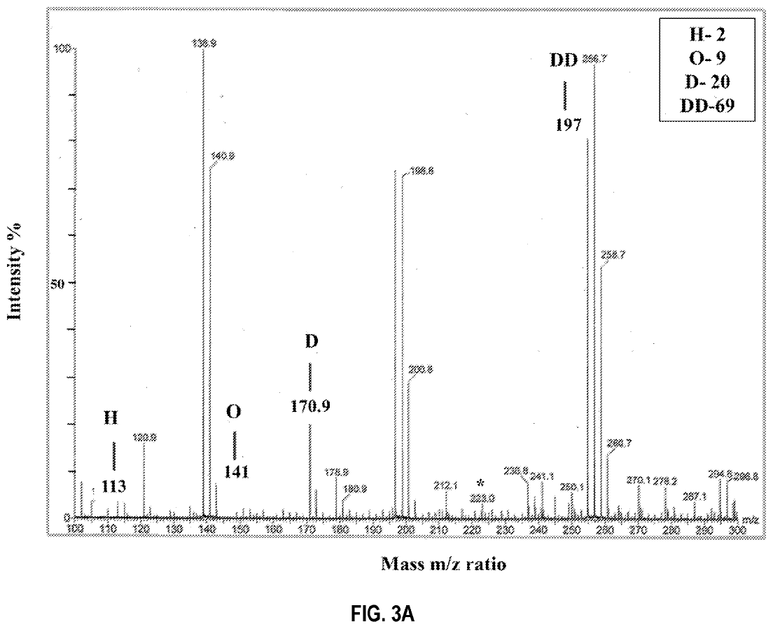

FTIR analysis further supported the observation of PHA accumulation, with a signature band characteristic of the C═O group present in the range 1720 cm⁻¹-1740 cm⁻¹, with its exact position (reflecting its amorphous/crystalline state) being dependent on the extraction protocol and subsequent processing conditions (FIG. 2A-B). Further characterization has shown that the PHA being produced by Geobacillus thermodenitrificans strain cnambio1 is a high molecular weight.

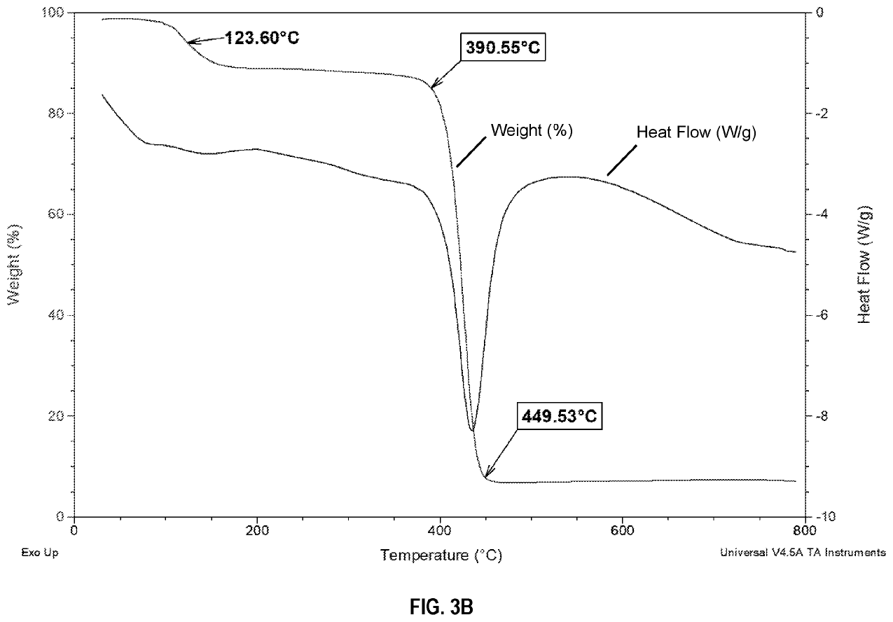

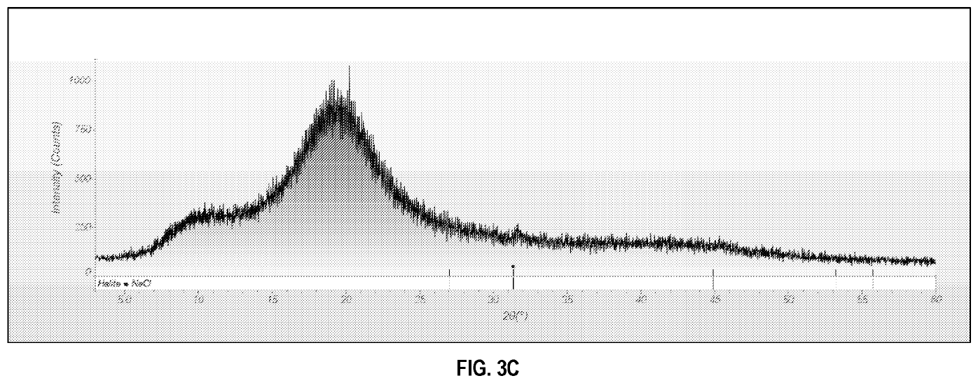

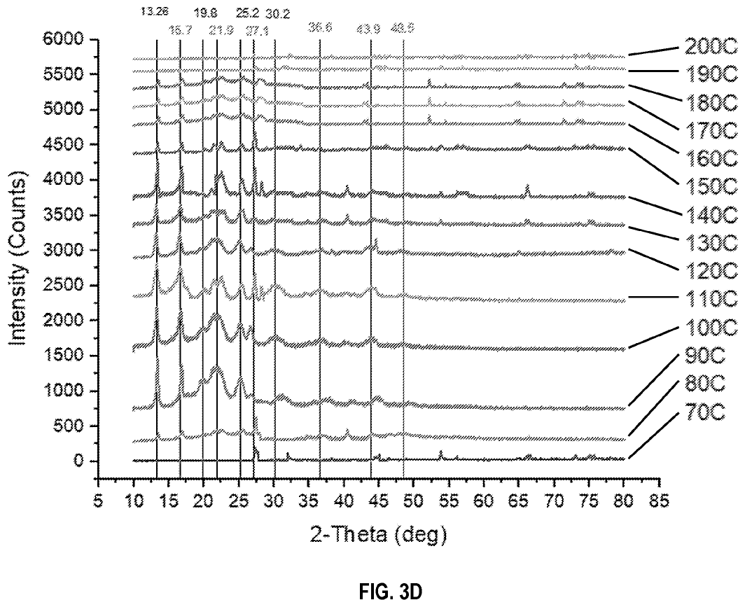

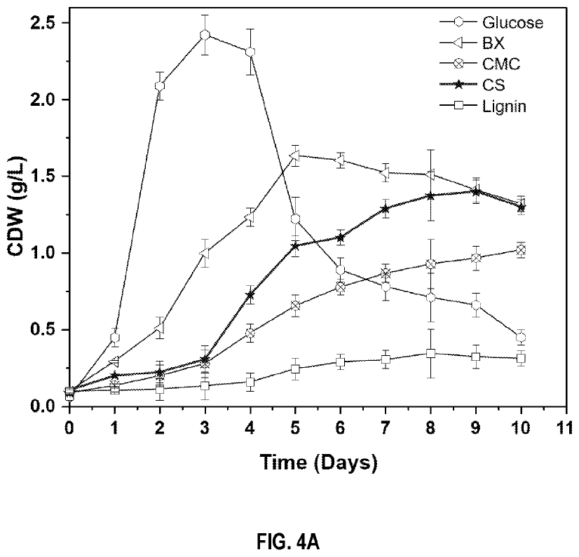

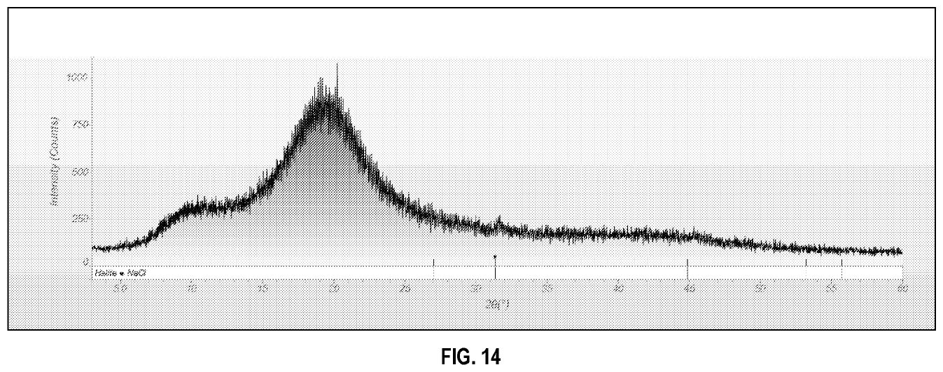

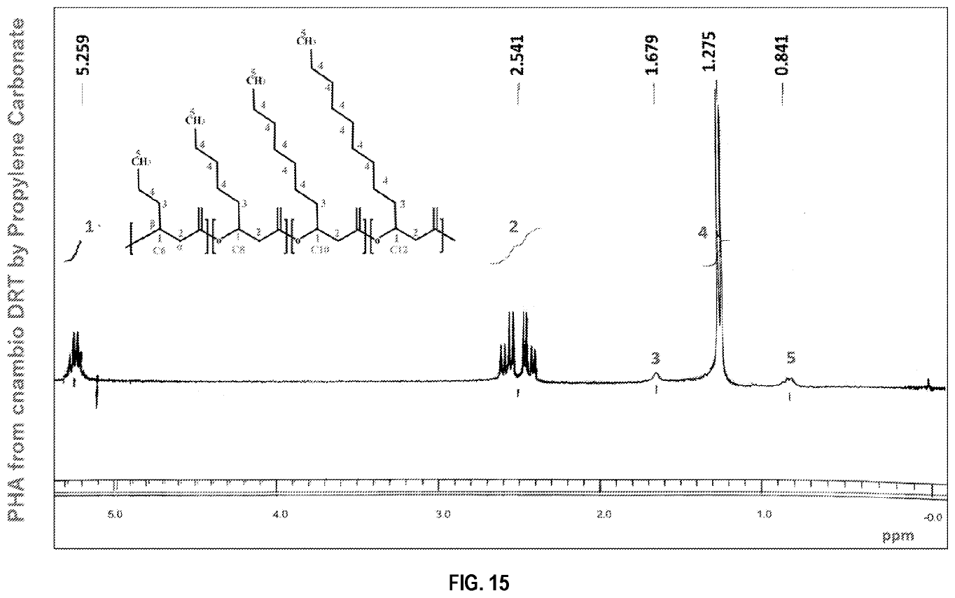

While simultaneously expressing its class IV PHA synthases (revealed by its whole genome analysis), Geobacillus thermodenitrificans strain cnambio1 ferments the sugars into a high molecular medium chain length, semi-crystalline to amorphous PHA (FIG. 3A) that has exceptionally high thermostability, with a thermal degradation temperature as high as about 425° C.-450° C. (FIGS. 3B, 7B and 13B) and a degradation onset temperature of about 390° C. (FIGS. 3B and 13B), Interestingly, such a high thermostability has not been reported so far for any PHA. We compared our results with those reported in literature, which are provided in Table 2 below.

TABLE 2

| | | | Degradation | |
|---|---|---|---|---|
| | Polymer | PHA | Temp. | |
| Bacteria | Composition | Category | (° C.) | Source |
| Geobacillus | 3HHx, 3HO, 3HD, | mcl | 420 | This |
| thermodenitrificans | and 3HDD | | | disclosure |
| strain cnambiol | | | | |
| Geobacillus | 3PHB | scl | 328.3 | Gedikli |
| kaustophilus | | | | et al., 2019 |
| Geobacillus sp., | 3PHB | scl | 280 | Giedraityte |
| AY846034 strain | | | | et al., 2015 |
| Aneurinibacillus | P(3HB-co-4HB-co- | scl | 280 | Sedlacek |
| sp. H1 | 3HV) | | | et al., 2020 |
| Pseudomonas | 3HHx (3.54 mole %); | mcl | 318.8 | Gumel et |
| putida Bet001 | 3HO (38.19 mole %); | | | al., 2012 |
| | 3HD (38.85 mole %); | | | |
| | and 3HDD (19.42 | | | |
| | mole %) | | | |

References cited
Gedikli, S., Çelik, P. A., Demirbilek, M. et al. Experimental Exploration of Thermostable Poly (β-Hydroxybutyrates) by Geobacillus kaustophilus Using Box-Behnken Design. J Polym Environ 27, 245-255 (2019).
Giedraityte G, Kalediene L (2015) Purification and characterization of polyhyroxy butyrate produced from thermophilic Geobacillus sp. AY 946034 strains. Chemija 26: 38-45.
Sedlacek, P., Pernicova, I., Novackova, I., Kourilova, X., Kalina, M., Kovalcik, A., Koller, M., Nebesarova, J., Krzyzanek, V., Hrubanova, K., Masilko, J., Slaninova, E., Trudicova, M., & Obruca, S. (2020). Introducing the Newly Isolated Bacterium Aneurinibacillus sp. H1 as an Auspicious Thermophilic Producer of Various Polyhydroxyalkanoates (PHA) Copolymers-2. Material Study on the Produced Copolymers. Polymers, 12(6), 1298.
Gumel A M, Annuar M S M, Heidelberg T (2012) Biosynthesis and Characterization of Polyhydroxyalkanoates Copolymers Produced by Pseudomonas putida Bet001 Isolated from Palm Oil Mill Effluent. PLOS ONE 7(9): e45214.

Detailed studies of the extracted mcl-PHA using XRD show it to be an amorphous polymer (FIG. 3C), capable of slow, thermal crystallization when heated at temperatures between 80° C. and onset of melting (FIG. 3D). The onset of viscous flow in the amorphous polymers starts at about 210° C. As is well known in the art, the crystallization rate would be expected to increase strongly in the presence of significant preferred orientation of the PHA molecules (e.g., resulting from fiber spinning/drawing and other methods of stretching the polymer).

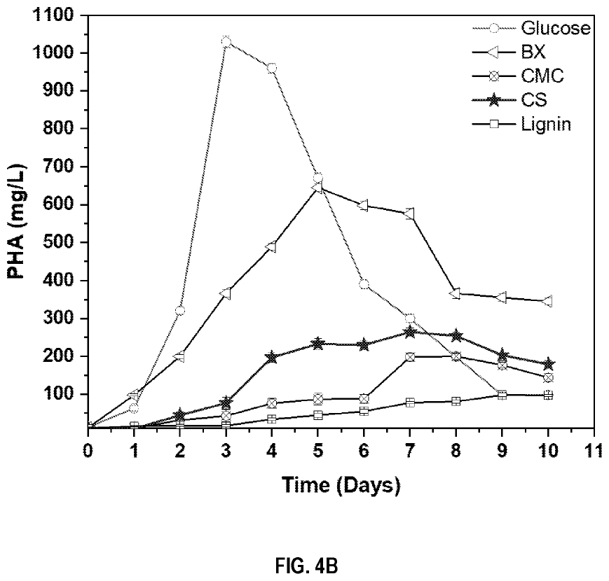

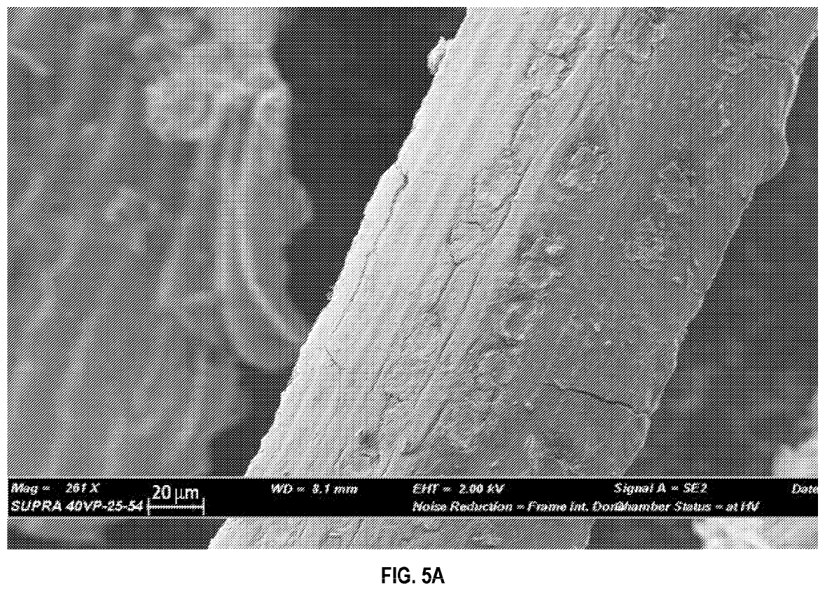

Geobacillus thermodenitrificans strain cnambio1 was found to grow well on various cellulosic materials (including microcrystalline cellulose) and hemicellulosic substrates e.g., Beechwood Xylan (FIG. 4A-B). It also grows and produces PHAs with Kraft lignin (98 mg/L) as the sole carbon and energy source in the medium. Natural lignocellulosic materials (e.g., corn stover) without any physicochemical or biological pretreatment also supports appreciable growth of Geobacillus thermodenitrificans strain cnambio1.

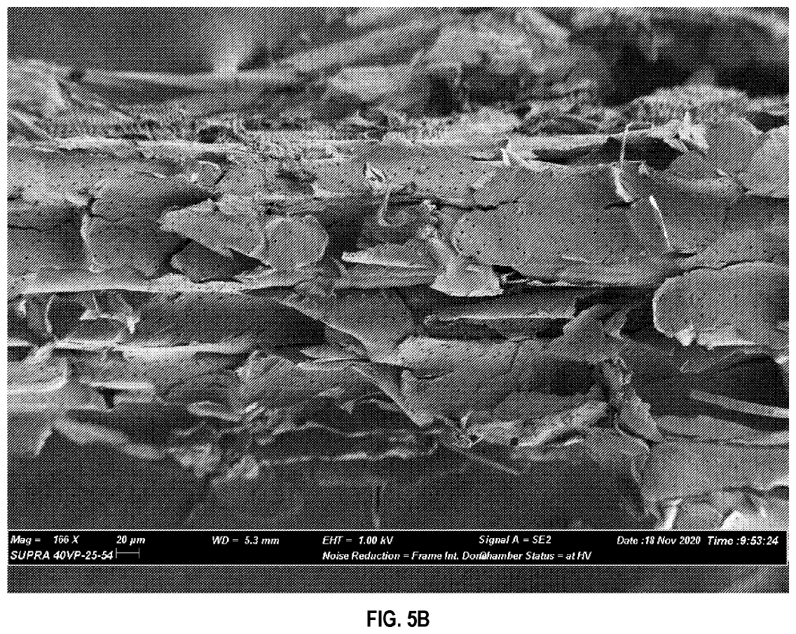

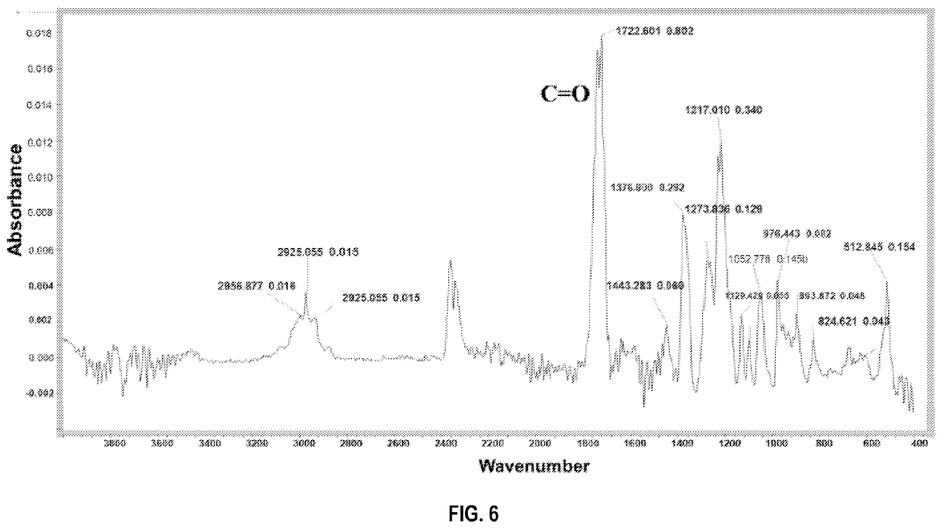

To evaluate the lignocellulose degradation efficiency of Geobacillus thermodenitrificans strain cnambio1, the chemical composition of the corn stover (CS) was analyzed by treating 0.5% (w/v) of the substrate with 5% (v/v) inoculum of the respective strain for 10 days at 60° C., pH 7.0, and agitation speed of 150 rpm. The results of the chemical composition analysis demonstrate that when used as a biocatalyst (at 5% w/v inoculum), Geobacillus thermodenitrificans strain cnambio1 can reduce the original lignin, hemicellulose, and cellulose fractions, relative to unprocessed corn stover, by 19.3%, 15.4%, and 4.6%, respectively. Furthermore, a qualitative SEM analysis, employed to give some insight into the structural modifications of corn stover after microbial treatment, provide clear evidence of corrosion of the corn stover samples that were biotreated with Geobacillus thermodenitrificans strain cnambio1 (FIG. 5B) compared to the relatively smooth and flat surface of the untreated corn stover (FIG. 5A).

Geobacillus thermodenitrificans strain cnambio1
16S rRNA, small subunit ribosomal RNA
(SEQ ID NO: 1)
tttcttttggagagtttgatcctggctcaggacgaacgctggcgg
cgtgcctaatacatgcaagtcgagcggaccgaacgagagcttgct
cttgttcggtcagcggcggacgggtgagtaacacgtgggcaacct
gcccgcaagaccgggataactccgggaaaccggagctaataccgg
ataacaccaaagaccgcatggtctttggttgaaaggcggcttcgg
ctgtcacttgcggatgggcccgcggcgcattagctagttggtgag
gtaacggctcaccaaggcgacgatgcgtagccggcctgagagggt
gaccggccacactgggactgagacacggcccagactcctacggga
ggcagcagtagggaatcttccgcaatggacgaaagtctgacggag
cgacgccgcgtgagcgaagaaggccttcgggtcgtaaagctctgt
tgtgagggacgaaggagcgccgtttgaataaggcggcgcggtgac
gg-acctcacgagaaagccccggctaactacgtgccagcagccgc
ggtaatacgtagggggcgagcgttgtccggaattattgggcgtaa
agcgcgcgcaggcggtcctttaagtctgatgtgaaagcccacggc
tcaaccgtggagagtcattggaaactgggggacttgagtgcagga
gaggagagcggaattccacgtgtagcggtgaaatgcgtagagatg
tggaggaacaccagtggcgaaggcggctctctggcctgtaactga
cgctgaggcgcgaaagcgtggggagcaaacaggattagataccct
ggtagtccacgccgtaaacgatgagtgctaagtgttagaggggtc
acaccctttagtgctgtagctaacgcgataagcactccgcctggg
gagtacggccgcaaggctgaaactcaaaggaattgacgggggccc
gcacaagcggtggagcatgtggtttaattcgaagcaacgcgaaga
accttaccaggtcttgacatcccctgacaacccaagagattgggc
gttccccttcggggggacagggtgacaggtggtgcatggttgtcg
tcagctcgtgtcgtgagatgttgggttaagtcccgcaacgagcgc
aacccttgcctctagttgccagcattcagttgggcactctagagg
gactgccggctaaaagtcggaggaaggtggggatgacgtcaaatc
atcatgccccttatgacctgggctacacacgtgctacaatgggcg
gtacaaagggctgcgaacccgcgagggggagcgaatcccaaaaag
ccgctctcagttcggattgcaggctgcaactcgcctgcatgaagc
cggaatcgctagtaatcgcggatcagcatgccgcggtgaatacgt
tcccgggccttgtacacaccgcccgtcacaccacgagagcttgca
acacccgaagtcggtgaggtaacccttacgggagccagccgccga
aggtggggcaagtgattggggtgaagtcgtaacaaggtagccgta
ccggaaggtgcggctggatcacctcctttcta

### Example 2: Optimized Growth Conditions for Treating Unprocessed Lignocellulosic Biomass and for Producing Polyhydroxyalkanoate (PHA) from Biomass

### Conditions Suitable for Growth of the Strain (Preparing the Inoculum)

The bacterium Geobacillus thermodenitrificans strain cnambio1 isolated from Landfill compost Facility of South Dakota was purified and maintained in an agar plate at 4° C. For the inoculum, the bacterium was grown in a conical flask containing Luria broth medium for 24 h, at 60° C., pH 6.8, with a rotation of 180 rpm and stored at 4° C.

Conditions Suitable for Treating Unprocessed (without Pretreatment) of the Lignocellulosic Biomass

The growth conditions were optimized for treating lignocellulosic biomass:temperature 60° C., pH 6.8, rotation of 200 rpm, inoculum volume of 7% v/v and a minimal media with the composition (for 1 L media) as listed in Table 3. All the carbon sources (substrates) were supplemented to the medium at a specified concentration, as mentioned in Table 4. Then the media was finally inoculated with 7% v/v of bacterial inoculum and maintained at the optimized growth conditions. Table 3 shows minimal media composition for the depolymerization of unprocessed agri-wastes by Geobacillus thermodenitrificans strain cnambio1. Table 4 shows substrate concentration for the depolymerization of unprocessed agri-wastes by Geobacillus thermodenitrificans strain cnambio1.

TABLE 3

| Chemical | Amount (in g) |
|---|---|
| Potassium nitrate | 0.25 |
| Monopotassium phosphate | 0.1 |
| Magnesium sulphate | 0.1 |
| Yeast extract | 0.5 |
| Sodium molybdate | 0.05 |
| Calcium chloride | 0.02 |

TABLE 4

| Substrate | Concentration | |
|---|---|---|
| Corn stover | 0.5% | (w/v) |
| Beechwood xylan | 0.5% | (v/v) |
| Carboxymethyl cellulose | 0.1% | (v/v) |
| Glucose | 2.0% | (v/v) |
| Lignin | 0.025% | (v/v) |

Producing a Polyhydroxyalkanoate (PHA) from Biomass

The growth conditions were optimized for producing PHA from unprocessed biomass (Agri-waste) at temperature 60° C., pH 6.8, rotation of 200 rpm, inoculum volume=7% and a minimal media with the composition (for 1 L media). Table 5 shows a minimal media composition for the fermentation of unprocessed agri-wastes to Polyhydroxyalkanoates (PHA) by Geobacillus thermodenitrificans strain cnambio1.

TABLE 5

| Chemical | Amount (in g/L) |
|---|---|
| Potassium nitrate | 0.25 |
| Monopotassium phosphate | 2 |
| Magnesium Sulphate heptahydrate | 0.5 |
| Yeast extract | 0.1 |
| Sodium molybdate | 0.05 |
| Calcium chloride dihydrate | 0.02 |
| Sodium bicarbonate | 0.5 |
| Corn Stover | 5 |

### Example 3: Characterization of PHA Extracted from Geobacillus thermodenitrificans Strain cnambio1

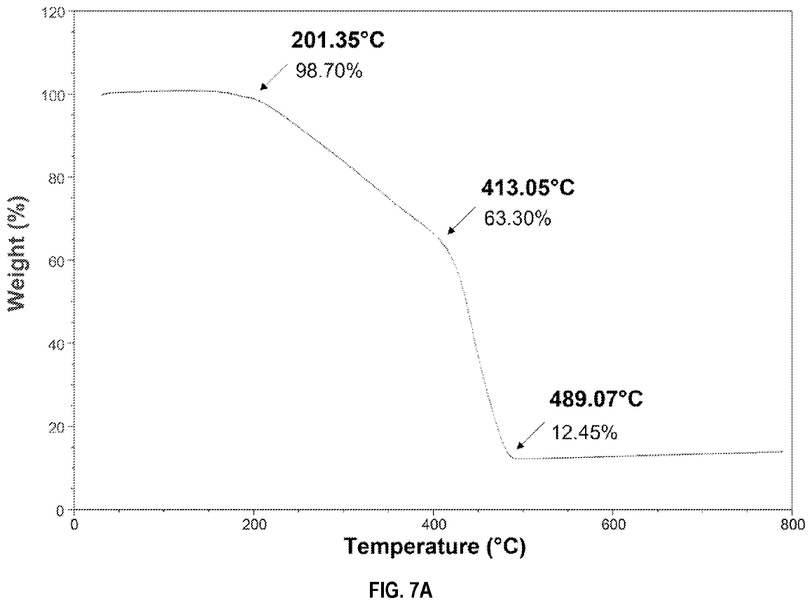

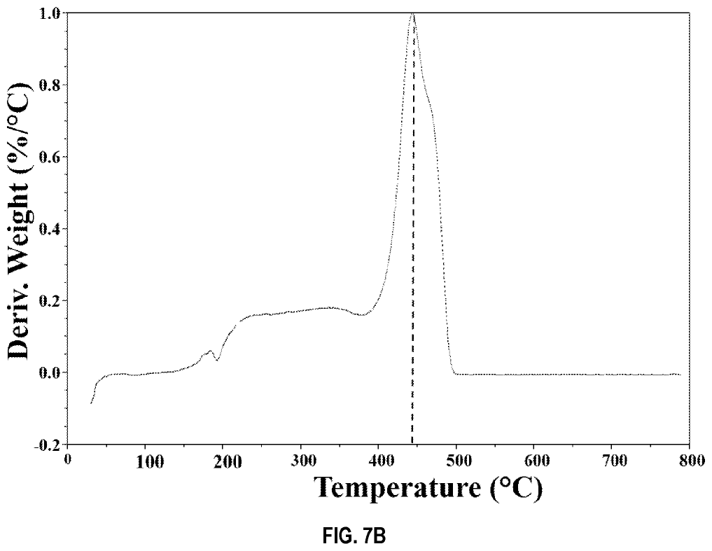

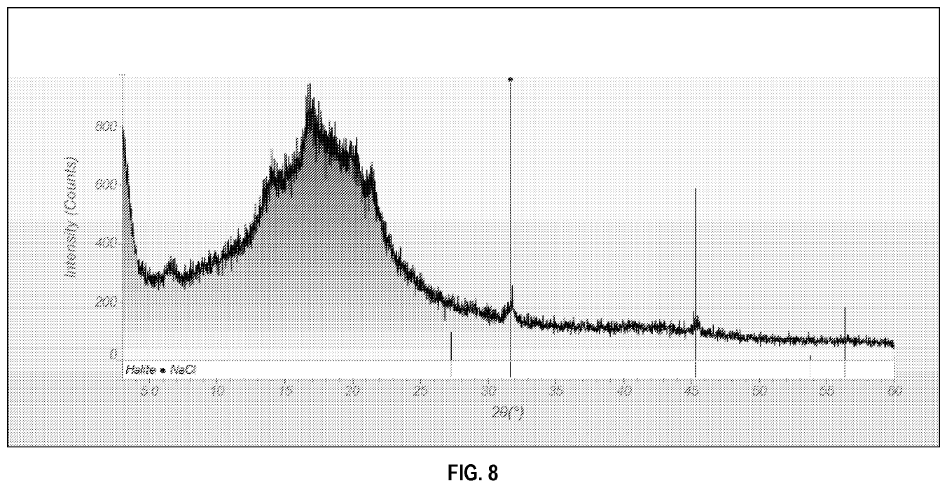

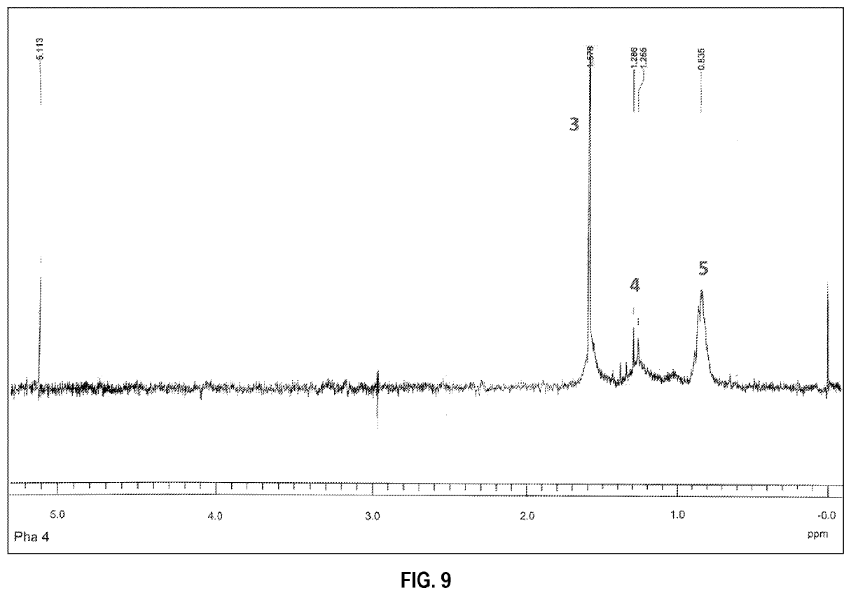

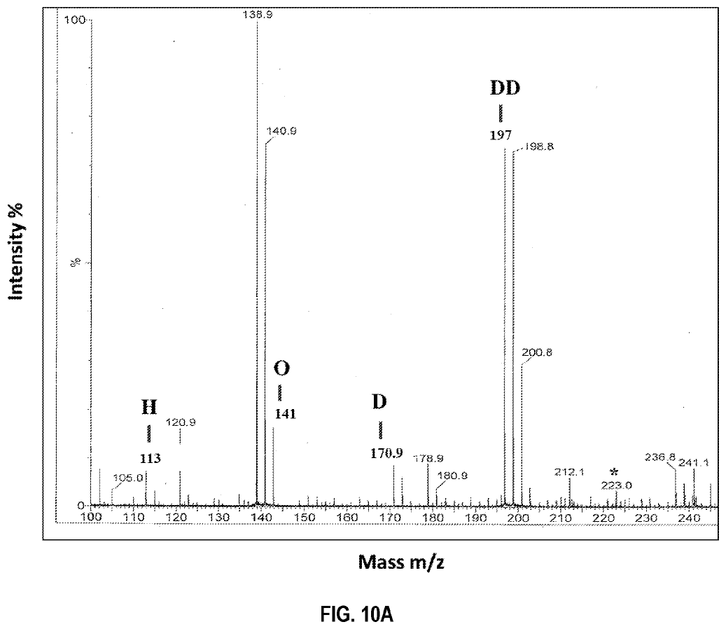

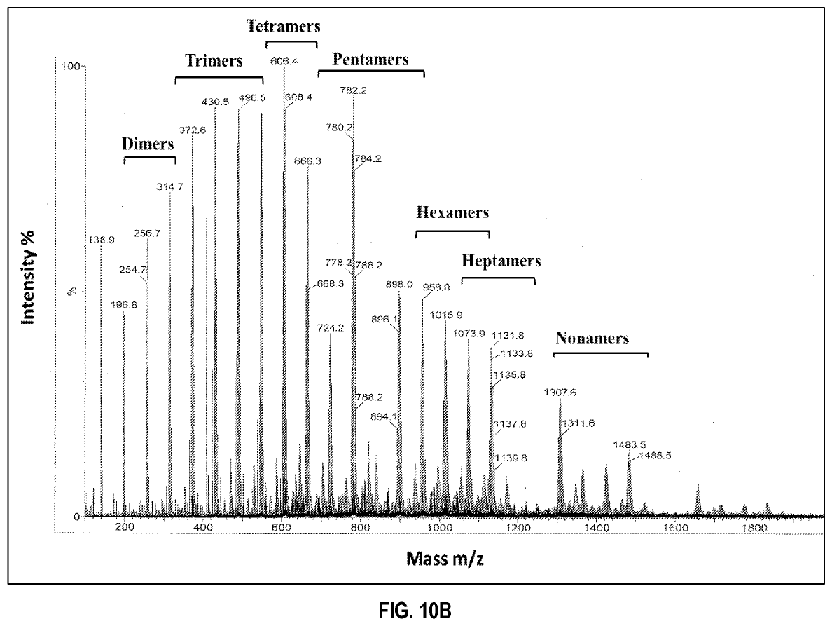

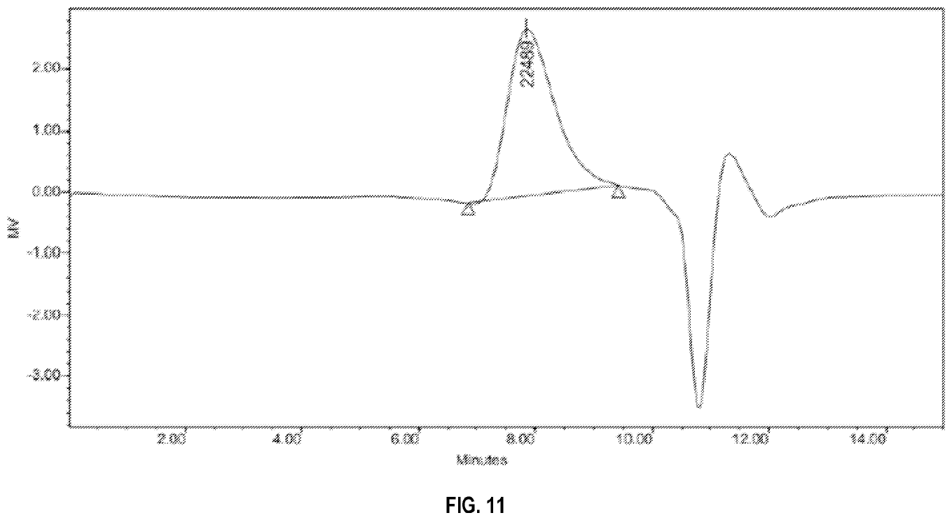

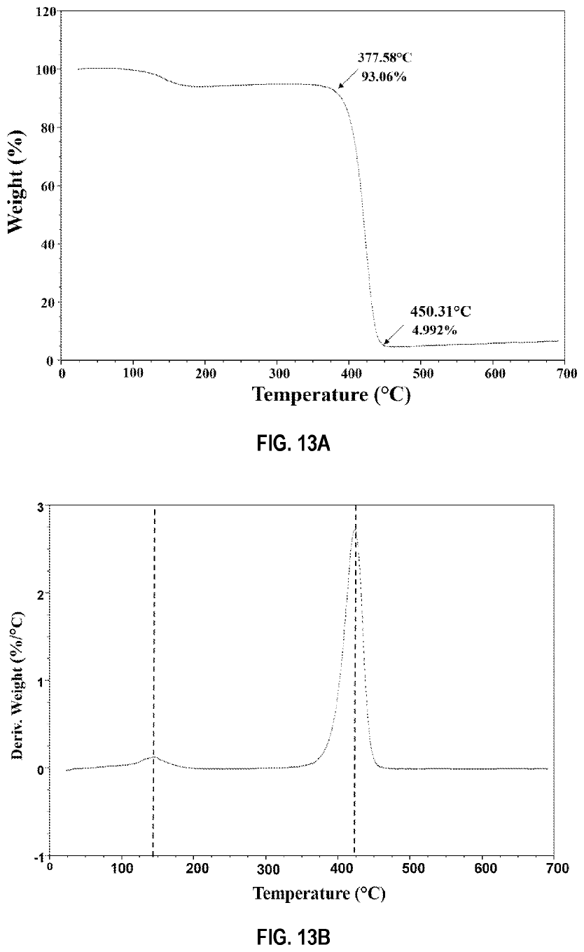

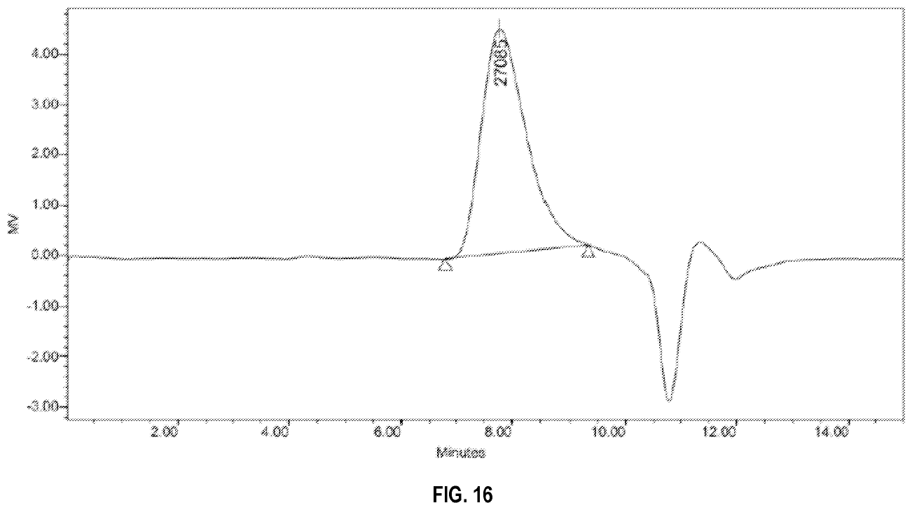

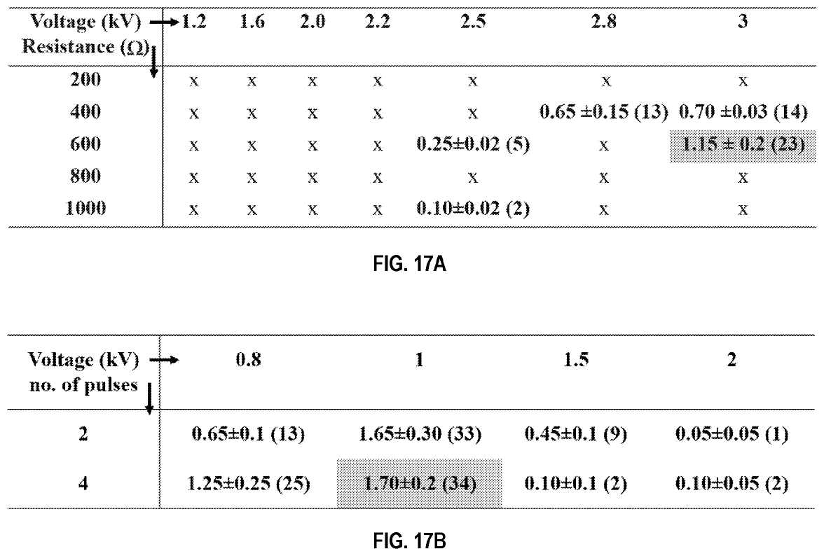

PHA was extracted from Geobacillus thermodenitrificans strain cnambio1 using either chloroform or propylene carbonate. The extracted PHAs were characterized by Fourier-transform infrared spectroscopy (FIG. 6, FIG. 12), thermogravimetric analysis (FIG. 7, FIG. 13), X-ray diffraction (FIG. 8, FIG. 14), nuclear magnetic resonance (FIG. 9, FIG. 15), and MALDI-TOF mass spectra (FIG. 10).

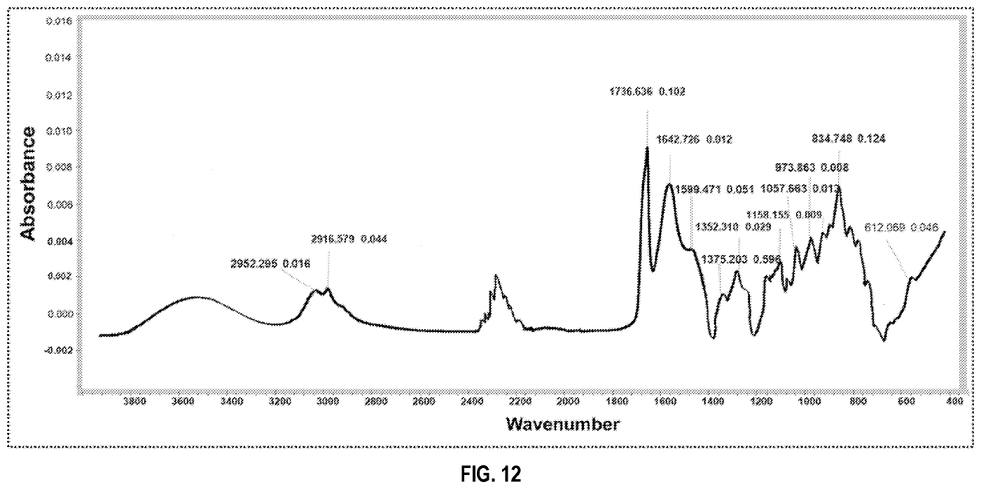

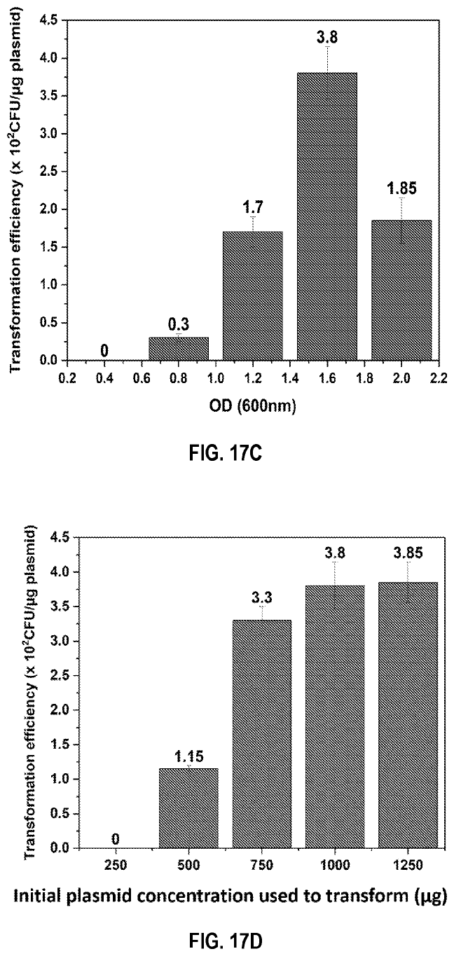

Molecular weight distribution of the PHA extracted from Geobacillus thermodenitrificans strain cnambio1 using chloroform (FIG. 11, Table 6) or propylene carbonate (FIG. 16; Table 7) was determined using the GPC waters 1515-2414 system.

TABLE 6

| Mw (g/mol) | Mn (g/mol) | Mw/Mn (PDI Index) |
|---|---|---|
| 23658 | 10672 | 2.217 |

TABLE 7

| Mw (g/mol) | Mn (g/mol) | Mw/Mn (PDI Index) |
|---|---|---|
| 28721 | 13142 | 2.151 |

### Example 4: Development of the Genetically Engineered Strains of cnambio1

The amount of PHA produced with corn stover is currently 402 mg/L (about two-fold lower than with glucose). We anticipate that genome engineering of Geobacillus thermodenitrificans strain cnambio1 can be performed to increase the yield of PHA in Geobacillus thermodenitrificans strain cnambio1.

### Enhance Cellulose Hydrolysis by Improved Cell-to-Cellulose Interactions

Cellulase enzymes will be displayed on cell surfaces of Geobacillus thermodenitrificans strain cnambio1 using protein and cell surface engineering. For this purpose, the GE40 protein (UniProt ID: A0A291I5R3) from Geobacillus thermodenitrificans T12 will be used as the cellulase. Herein, linker and the peptidoglycan-binding domain (PBD) of the lysin (UniProt ID: W8EEW7: 124-214 aa) found in the phage GBK21, which infects the Geobacillus spp. will be used as the anchor protein to display GE40 on the cell surface of Geobacillus thermodenitrificans strain cnambio1. Plasmid pG1AK containing thermophilic (thermostable at 60° C.) super-folding green fluorescent protein marker (GFP) (pG1AK-sfGFP, Addgene) will be used as the vector backbone for transforming the genes in Geobacillus thermodenitrificans strain cnambio1. The constructed recombinant plasmid, pG1AK-PBD-GE40, will be transformed into the host Geobacillus thermodenitrificans strain cnambio1 by electroporation.

>tr|A0A29115R3|A0A29115R3_GEOTD GE40
OS = Geobacillus thermodenitrificans
OX = 33940 GN = ge40 PE = 3 SV = 1
(SEQ ID NO: 2)
MKRIGRIFLCAMLFAYVFFAGKPLLSKAEDNKASAYDINEYANSM
QPGWNLGNTFDGFDTGKIVLDETAWGNPRVTKELIDKIADEGFKS
IRIPITFDTRLSDGPDYTINPEELARIERVVNWALEANLKVMINI
HHDSWRWIADGMVHDHDNTVAKFKAIWTQLADRFKDYNLDLMFES
LNEPQFWGAPEDSQRYLNELNSLFYSIVRHSGGNNDIRPLVLPTL
NTGSEPEKLDALYNFITQLNDPYIIATIHYYGFWPFSVNIAGVTN
FNEETKNDIIHAFDRVHDKFIKNGIPVVIGEYGLLGFDRGTGTIQ
QGEKLKYFEFMIHYAQEKNLIHMLWDNGQHFGRTSFKWYDAEFGE
MLKASWNGRSATADANFIYLKQGKPVQDVTRILQLNGRKFISLQL
NGKDLVAGKDYEINGKSLTIKASLLSKLVSNKIREKAVLTATEDK
GANWYFHIFNYDTPSLSDSTGTVSNFTIPIKENGTHLKTMEAKYV
DDGSNAGPQNWTSFKEFGYAFSPDYEHDVVTFPYGNERFFLKMAG
K

### PHA Yield Enhancement Via Knockout of Pha Depolymerase (PhaZ) Enzyme to Remove the PHA Degradation Mechanism

Construction of the phaZ mutant of Geobacillus thermodenitrificans strain cnambio1 (ΔphaZ) will be performed using homologous recombination by ligating three fragments: upstream of the phaZ, kanamycin marker, downstream of phaZ (assembled using Gibson assembly) into the selective plasmid pG2K. Correct assembly of fragments will be verified using restriction digests and sequencing. Once verified, the recombinant plasmid will be transformed into Geobacillus thermodenitrificans strain cnambio1 by electroporation. Deletion will be verified by diagnostic PCR and qPCR using locus-specific primers.

Geobacillus thermodenitrificans strain
cnambio1 PhaZ PHA depolymerase protein
sequence
(SEQ ID NO: 3)
MVIIEKETVAQVPVLHVVKKEKREERLPFILFIHGETSAKEHNLH
EGYLLAEAGYRVILPDALHHGERDSSLSERELQLAFWNIVTRTIT
EIKKIKEELELRNLIQPDRIGLAGTSMGGIVTFGALAEYPWIKAA
VSLMGNPMYEAFFDALIETGKKMGVAIPLSDEQLKREKARLMKYD
LSRQPEKLAGRPLLIWHGKCDQVVPYSYTYEFYEQIKPLYQGKEE
NLKFISDDTAGHKVTREAFLETVKWFTKHV
Geobacillus thermodenitrificans strain
cnambio1 PhaZ PHA depolymerase nucleotide
sequence
(SEQ ID NO: 4)
atggtcatcattgaaaaagaaaccgttgcccaagtgcctgtgctt
catgtcgtaaaaaaagaaaagcgggaagaacgattgccgtttatt
ttgtttattcatggatttacaagtgcaaaagaacataatttgcac
tttgggtacttgcttgccgaggcgggataccgcgtgattttgccg
gatgcgctgcatcacggtgaacgagattcgtctttatctgagcgt
gagctgcaattggcgttctggaatattgtgacgcgtacgattacg
gaaattaaaaaaatcaaagaggaattagaacttcgcaatctcatt
cagccggatcggataggacttgccggtacatcgatgggggggatt
gtcacatttggcgcacttgccgagtatccatggattaaagcggcc
gtttcgctgatgggaaatccaatgtatgaagcattttttgacgcc
ttaattgaaacaggcaaaaaaatgggagtggcgattccgctttcc
gatgaacaactaaagcgggaaaaggcgcgattaatgaaatatgac
ctttctagacagccggagaaattagcgggccgtccgcttcttatt
tggcatggaaagtgcgaccaagttgtcccttattcttatacatat
gagttttatgaacaaataaaaccgctttatcaaggaaaagaagaa
aatttgaagtttatttccgatgatacggcagggcataaagtgaca
agggaagcgtttttagaaacggtgaaatggtttacgaaacatgtg
taa

### Overexpress PHA Synthesis Operon (PhaCAB) to Improve PHA Productivity

The native promoters of some specific genes of PHA biosynthetic operon (Class IV) in Geobacillus thermodenitrificans strain cnambio1 will be replaced with the strong constitutive modified promoter that will be sensitive to the presence of the hexose and the pentose sugars. The same homologous recombination-based gene replacement strategy will be followed here. We expect that we will be able to further enhance the PHA concentration and accumulation percentage in Geobacillus thermodenitrificans strain cnambio1 by 2-4 fold and reduce the total fermentative time of PHA production.

Geobacillus thermodenitrificans strain
cnambio1 PhaC PHA synthase protein sequence
(SEQ ID NO: 5)
MPYIVEDGVRLYYEEMGSGTPILFIHPPGMGHIVFRHQQSLSSHF
RIIMYDMRGNGKSSPSNRPITIPLLADDICRLLNTLDVKQAIICG
YSSGGSIASEFALRYPHKVKKLILIGGESEVCTPLLRYEFLLGIY
AAKIGAISLLANVLAKSHEKNKKGQQEIKQYVRLVNKKDLVNMYE
KGLTYSCTERLPLLRMPILLIYGARDYYMHPYEKLERKNVPHAKI
IYIENGRHQIPTKHHCELNGILLKTYCIT
Geobacillus thermodenitrificans strain
cnambio1 PhaC PHA synthase nucleotide
sequence
(SEQ ID NO: 6)
ATGCCATATATTGTAGAAGATGGCGTTCGTCTTTACTATGAAGAG
ATGGGAAGCGGTACACCAATTTTATTCATTCATCCACCTGGAATG
GGTCATATCGTTTTCCGTCATCAACAATCTTTATCAAGCCACTTC
CGCATCATCATGTATGATATGCGCGGCAATGGGAAAAGCAGTCCG
TCGAATCGTCCGATTACGATTCCGCTGCTAGCTGATGATATTTGC
CGCTTACTCAATACACTTGACGTAAAGCAAGCGATTATTTGCGGC
TATTCTAGCGGTGGATCCATTGCGTTAGAATTTGCCTTACGCTAT
CCTCATAAAGTAAAAAAGCTCATTTTAATTGGCGGATTCTCTGAA
GTATGTACTCCTTTATTGCGTTATGAATTCTTGCTTGGCATTTAT
GCAGCAAAAATCGGCGCTATTTCTTTACTTGCCAACGTATTAGCC
AAATCGCATGAAAAAAATAAAAAAGGACAACAAGAAATAAAACAA
TATGTGCGATTGGTCAATAAAAAAGATTTAGTAAACATGTACGAA
AAAGGGTTAACATATTCCTGTACGGAGCGCCTTCCATTGTTGCGC
ATGCCAATCCTTCTTATTTATGGCGCCAGAGATTACTATATGCAT
CCATATGAGAAGCTGTTCAGGAAAAATGTCCCCCATGCGAAAATC
ATCTATATTGAAAACGGACGCCATCAAATTCCAACCAAACATCAC
TGCGAATTAAACGGGATTTTGAAGACATACTGTATAACGTAA
Geobacillus thermodenitrificans strain
cnambio1 PhaA 3-ketothiolase protein sequence
(SEQ ID NO: 7)
MREVVIVEAVRTPVGKRNGVFRNVHPVHLASTVLNEVVKRAGIEK
RLVEDIVMGCVTPIAEQGYNIGRLAALEAGFPIEVPAVQINRMCG
SGQQAIHFAAQEIRSGDMDITIAAGVESMTKVPILSDGNEKTIPP
SLHEKYEFVHQGISAELIAEKYGLTREQLDAYAYESHQRAIRAQE
QGIFDQEIVPVEGLDKEGN
Geobacillus thermodenitrificans strain
cnambio1 PhaA 3-ketothiolase nucleotide
sequence
(SEQ ID NO: 8)
ATGAGAGAAGTTGTCATTGTCGAAGCCGTGCGCACGCCAGTCGGC
AAGCGAAACGGCGTGTTCCGCAACGTACATCCCGTTCATTTAGCG
TCAACGGTGCTCAATGAAGTCGTAAAAAGAGCGGGAATCGAAAAA
CGGCTTGTCGAAGATATTGTGATGGGATGTGTCACGCCCATTGCA
GAGCAAGGATACAATATTGGGCGGCTTGCTGCGCTGGAGGCGGGA
TTTCCAATCGAAGTGCCAGCCGTGCAAATCAATCGGATGTGCGGG
TCAGGGCAGCAGGCAATTCATTTTGCTGCCCAAGAAATTCGCTCT
GGCGATATGGATATTACGATTGCTGCTGGCGTCGAAAGCATGACG
AAGGTGCCGATTTTAAGCGATGGAAACGAAAAAACGATTCCGCCG
TCGCTGCATGAAAAATATGAATTTGTTCATCAAGGCATTTCCGCG
GAATTAATCGCCGAAAAGTACGGGCTGACGCGCGAACAGCTGGAC
GCATATGCATACGAAAGTCATCAGCGCGCGATTCGGGCGCAAGAA
CAAGGAATATTTGATCAAGAAATCGTGCCTGTGGAAGGTTTGGAT
AAAGAAGGGAAC
Geobacillus thermodenitrificans strain
cnambio1 PhaB Acetoacetyl-CoA reductase
protein sequence
(SEQ ID NO: 9)
MEFGLAGKTALVAASSQGLGKAIARALVLEGANVMITSRNEEKLQ
EVAEELNSLHKGRVAYTRTDVTKADDIRQLVAKTVETFGTIDLLV
NNAGGPPAGTFETISDKDWQYAFELNLLSYIRLIREALPYLKKKG
GKIVNIASSSIKEPIPGLILSNTERTGIIGLTKTLATEFAPDNIL
INTVAPGRIATERVAFLDKVNAEKLGITKEEMEARMRSAIPLGRY
GTPEEFANVV
Geobacillus thermodenitrificans strain
cnambio1 PhaB Acetoacetyl-CoA reductase
nucleotide sequence
(SEQ ID NO: 10)
ATGGAGTTCGGATTGGCGGGAAAAACGGCGCTTGTCGCTGCATCG
AGCCAAGGGCTTGGCAAAGCGATTGCCCGAGCGCTTGTGCTGGAA
GGAGCAAACGTGATGATTACAAGCCGAAATGAGGAAAAGCTGCAG
GAAGTCGCCGAGGAGCTTAACAGTTTACATAAGGGACGCGTTGCT
TATACGCGCACCGATGTGACGAAAGCGGATGACATCCGCCAACTG
GTCGCCAAAACTGTGGAGACGTTTGGAACGATCGATTTACTTGTT
AACAACGCCGGCGGCCCTCCGGCGGGAACGTTCGAAACAATCAGC
GACAAAGACTGGCAATATGCGTTCGAGCTCAATTTACTGAGCTAT
ATTCGGTTGATTCGCGAAGCGTTGCCTTATCTAAAGAAAAAAGGC
GGTAAAATTGTCAATATCGCCTCGTCGTCCATCAAAGAGCCTATT
CCGGGGCTCATCCTGTCCAATACGTTCCGCACTGGGATCATCGGG
TTGACGAAAACGCTTGCAACGGAGTTCGCGCCTGATAATATTTTG
ATCAACACAGTCGCACCTGGACGGATTGCTACGGAACGGGTAGCT
TTCTTAGACAAGGTGAACGCTGAAAAGCTCGGCATTACGAAAGAA
GAAATGGAAGCGCGCATGAGAAGCGCCATTCCGCTCGGCCGTTAC
GGAACCCCTGAGGAGTTTGCCAACGTTGTC

### Example 5: Transforming Geobacillus thermodenitrificans Strain cnambio1

Every Geobacillus species or strain has its individual unique genetic and physiological characteristics that consequently demands specific conditions for optimal electro competency. Hence, this example aimed to establish reliable procedures of genetic transformation and genetic modification of Geobacillus thermodenitrificans strain cnambio1.

Preparing Electrocompetent Cells of cnambio1 Cells

For preparing electrocompetent cnambio1 cells, 5 ml seed culture of the strain was grown overnight at 55° C. in LB media with Glycine (0.75%), Twin-80 (0.05%), and sucrose (500 mM) (LGTS media). 1 ml of this seed culture was used, then inoculated into 100 ml of LGTS media, which was incubated at 55° C. with shaking at 220 rpm for 12-16 hours until the cells reached an OD_{600 nm} of 1.5-1.6. Individual experimental instances in with an OD_{600 nm} of 0.4, 0.8, 1.2, 1.6 or 2.0 were also performed. The cultures were kept on ice to chill for 20-30 minutes, with occasional swirling to ensure even cooling. The cells were harvested by centrifugation (15 min at 4000 rpm). The supernatant was decanted, and the cells were washed twice with ice-cold sterile deionized water, twice with 10% glycerol, and pelleted again by centrifugation. The cells were resuspended in 200 μL ice cold 10% glycerol and stored at −80° C. as 50 μL aliquots for electroporation until further use.

Transformation of Electrocompetent Geobacillus thermodenitrificans Strain Cnambio1

Foreign DNA (900-1000 ng of plasmid or linear DNA) was mixed with a 50 μL aliquot of ice-thawed electrocompetent cnambio1 cells and incubated on ice for 10 minutes. The DNA-cell suspensions were transferred to an ice-cold electroporation cuvette (BIO-RAD, USA). Four square wave pulses of 1 kV and 5 ms each were applied to the cuvettes using a Gene Pulser Xcell Microbial System (BIO-RAD, USA). This methodology was optimized by varying electric field (0.8, 1.0, 1.5, and 2.0) against the number of pulses (2 or 4). Individual experimental instances were also performed in which the exponential decaying pulse was set at different voltages (1,2, 1.6, 2.0, 2.2, 2.5, 2.8, or 3.0 kV) and resistances (200, 400, 600, 800, and 1000, 25 μF). A volume of 500 μL of 55° C. prewarmed LB media was added to the cuvette immediately after pulsing.

The mixture was transferred to a sterile 2 ml Eppendorf, and the cells were recovered at 55° C. for 3 hours with shaking at 200 rpm. Post incubation, the 100 μL cells were plated on pre-warmed (55° C.) LB agar plates containing appropriate selective antibiotics and incubated at 55° C. for 36-48 hours. The transformation efficiency was calculated by counting colony forming units, and using the following formula:

$$Transformation efficiency = \frac{Number of colonies counted ( CFU ’ s ) on plate}{Microgram ( μg ) of plasmoid / DNA used} \times \frac{Final volume at Recovery ( mL )}{Volume plated ( mL )}$$

The units for transformation efficiency is CFU/μg of exogenous plasmid.
Transformation Efficiency of Geobacillus thermodenitrificans Strain cnambio1

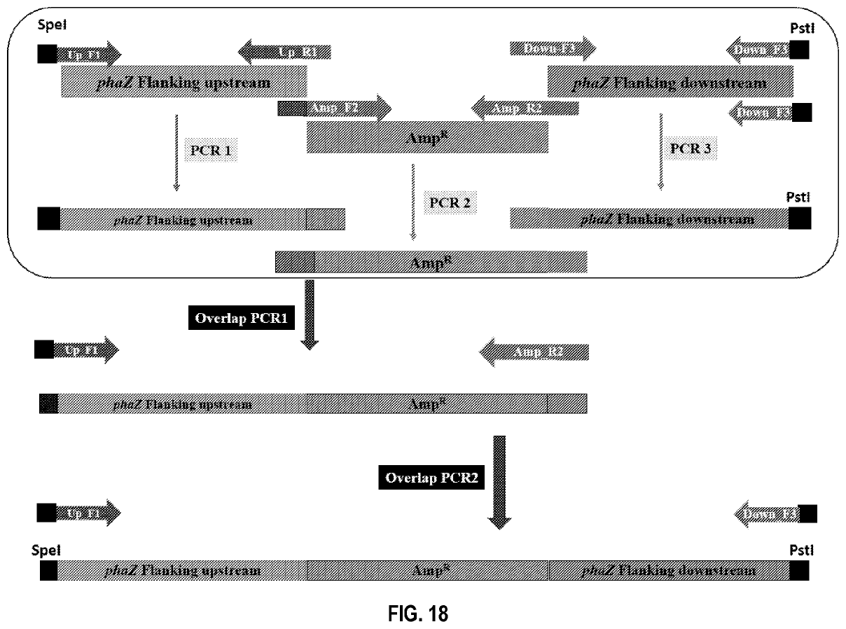

The E. coli-Geobacillus shuttle vector used in this example was PG2K (Addgene #71742; 3.8 kbp, repB replicon, Kan^{R}). The first optimization performed was to find the complementing conditions of electroporation voltage (kV) and electroporation resistance (Ω) that can generate maximum transformants for cnambio1 cells harvested and made electrocompetent at OD 1.2. The electric field can be applied to the cells as an exponential decay waveform pulse or as a square waveform pulse, both of which were tried for electroporating Geobacillus sp. strain cnambio1. The results (FIG. 17A) indicate that no transformation was obtained for cnambio1 cells when delivering the pulse as an exponential decay waveform at voltage values below 2.5 kV. Transformants were obtained at only particular combinations of kV and Q. The highest transformants of cnambio1 cells at 1.15±0.2×10² transformants μg⁻¹ PG2K plasmid were achieved at 3 kV and 600Ω for an exponential decay waveform. These findings are different from those reported in literature, where transformation efficiency has been shown to peak at 2.0-2.4 kV for other Geobacillus spp. including Geobacillus thermodenitrificans, with pulses above 2.4 kV being too lethal for the cells.

In the case of square waveform pulse, voltage was altered against the number of pulses provided to the electrocompetent cells of cnambio1, keeping length of each pulse at 5.0 ms. The highest transformants (1.70±0.2×10² transformants μg⁻¹ PG2K plasmid) were observed when applying 4 pulses, each of 1 kV electric field, and each lasting 5 ms (FIG. 17B). When applying electric field above 1 kV in square wave pulse with higher pulses, the number of transformants decreased rapidly. This may be explained by cell death but also by plasmid degradation arising during long pulses. With efficiency of transformation cnambio1 cells being approximately 30% higher with square wave pulses compared to exponential decaying applied, pulses conditions set at square waveform pulse were used for further experiments. Square wave electroporation system has never before been applied to any Geobacillus spp.

The second optimization was to find the culture age based on the OD₆₀₀ number at which cnambio1 cells were most electrocompetent. The different OD values tested represented different harvesting points in the log phase of the cnambio1 cells grown on LB media with LGST. When the cells attained the desired OD₆₀₀ number, cells were made competent using the protocol described above, and electroporated by a square wave pulse. The results (FIG. 17C) indicate that highest transformants of cnambio1 cells were achieved from the cultures harvested at OD₆₀₀ of 1.6.

Next, the effect of changing the concentrations of plasmids that were mixed with competent cells during electroporation was examined (with harvestable OD₆₀₀ at 1.5-1.6 and electroporation square wave conditions at 1 kV, 4 pulses, each of duration 5 ms fixed). The results indicated that transformation efficacy had a positive correlation with amount of plasmid used for electroporation (FIG. 17D). The highest transformation efficiency was achieved with plasmid concentrations reaching 1-1.25 μg. Adding DNA/plasmid beyond 1.25 μg caused arcing in the electroporation cuvettes, probably due salts or any other impurities in the DNA causing inability to transform the cells. Hence, a plasmid concentration of 900-1000 ng was used on the subsequent experiments.

Overall, this example demonstrates the ability to electroporate strain cnambio1 for the first time and identified factors that tremendously improve the transformation efficiency of this strain. The best results were achieved by growing cnambio1 cells to OD₆₀₀ of 1.5-1.6, mixing them with approximately 1000 ng of plasmid sample, and applying either a single exponential decaying electroporation pulse at 3 kV, 600Ω, and 25 μF, or four consecutive square wave electroporation pulses of 1 kV, 5 ms duration. Utilizing these conditions, a transformation efficiency of 3.85 0.7×10² CFU/μg plasmid (80 colonies per μg plasmid) was achieved with high reproducibility.

### Example 6: Creating phaZ Gene Knockout

Gene deletion by the overlap extension PCR dependent homologous recombination (HR) is a well-known standard, simple, and convenient method that has been widely adopted to create mutants in species or strain for which advanced genome editing methods are unavailable or underdeveloped. Several examples in literature describe employing HR as a method to knock out or replace chromosomal genes in genus Bacillus and Geobacillus. To generate a deletion gene cassette, the methodology demands, first, preparing an insertion segment (a segment or whole gene that will replace the target gene in the genome), and two 800-1000 bp long flanking fragments (upstream and downstream) of the gene to be deleted by three independent PCRs, using the appropriate primer pairs. The insertion segment has a region of 10 bp on both ends that overlap with the upstream and downstream flanking segments of the genes. Next, in the second step, the insertion and flanking fragments are stitched or annealed together to generate the final product. Strains and plasmids used in this example are summarized in Table 9.

TABLE 9

| | | |
|---|---|---|
| Strain or | | |
| plasmid | Description | Source |

E. coli strains

| | | |
|---|---|---|
| DH5alpha | hsdR17 (rk⁻, mk⁺) | ThermoFischer Scientific |
| BL21 (DE3) | dcm hsdSB (r_{B}⁻ m_{B}⁻) | ThermoFischer Scientific |
| INV110 | tsx dam dcm Δ(mcrCmrr) | ThermoFischer Scientific |
| TOP 10 | mcrA Δ(mrr-hsdRMS-mcrBC) | ThermoFischer Scientific |

Geobacillus thermodenitrificans strains

| | | |
|---|---|---|
| Cnambiol | Wild type | Rapid City Landfill |
| | | Compost |
| Cnambiol | phaZ Knockout strain | This example |
| Mutant | | |

Plasmids

| | | |
|---|---|---|
| PG2K | Temperature-sensitive suicide | Addgene |
| | plasmid, repB, Kan^{R} | |
| PG2K-phaZ | Containing phaZ deletion | This example |
| deletion | cassette of G. | |
| | thermodenitrificans | |
| | strain cnambiol | |

Abbreviations:
ATCC: American Type Culture Collection;
Kan^{R}: Kanamycin resistance gene

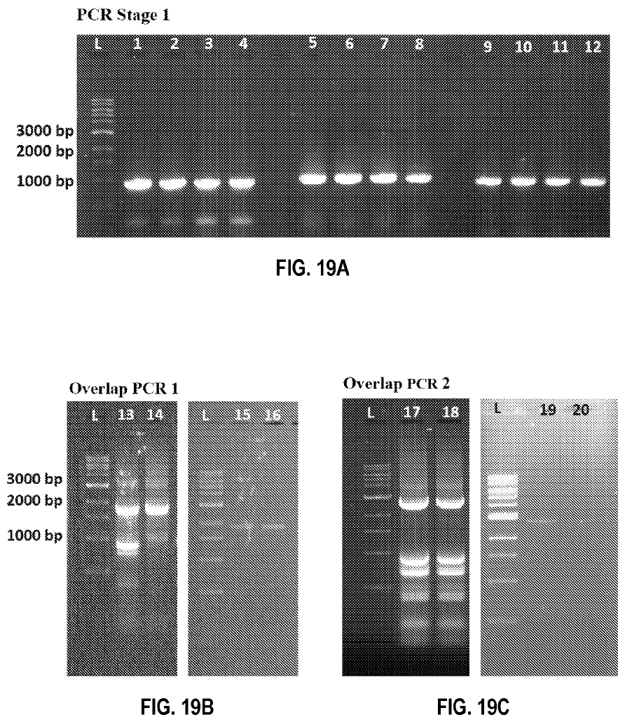

The 870 bp upstream and 800 bp downstream region of phaZ (the gene to be deleted) were used as the two flanking segments. The 960 bp Amp^{R} gene (the marker gene to replace the PhaZ gene) from plasmid PG1AK was used as the insertion segment. In the first stage of the PCR, these three fragments (i.e., phaZ upstream flanking, phaZ downstream flanking, and Amp^{R} gene) were amplified using cnambio1 genome, PG1AK plasmid, and cnambio1 genome as the respective templates, and up_F1/Up_R1, Amp_F2/Amp_R2, and Down_F3/Down_R3 as the respective primer pairs (FIG. 18). The two outer primers Up_F1, and Down_R3 had at their 5′ ends the two RE sites (SpeI, and PstI) to complement the MCS region of PG2K plasmid. The four inner or SOEing primers (i.e., Up_R1, Amp_F2, Amp_R2, and Down_F3) harbor the 10 bp overlap sequences complementary to each other. The oligonucleotides and probes used are summarized in Table 10.

TABLE 10

| | | | | |
|---|---|---|---|---|
| | Gene | | | Size of the |
| | location/ | Forward | Reverse | amplified |
| Gene | source | Sequence | Sequence | product |

Overlap PCR primers

| | | | | |
|---|---|---|---|---|
| phaZ | Cambio1 | Up_F1: | Up_R1: | 876 bp |
| flanking | DNA | TAAGCAACTAGTTTC | GGGTTCCGCGTGTTATA | |
| upstream | | GTTGTTGTCACATCGT | CAAACCCCTTTTCCTTT | |
| | | CAGCC | TCATG | |
| | | (SEQ ID NO: 11) | (SEQ ID NO: 12) | |
| Amp^{R} | PG1AK | Amp_F2: | Amp_R2: | 960 bp |
| | plasmid | TTGTATAACACGCGG | CCGACTTTGCTTACCAA | |
| | Addgenc | AACCCCTATTTGTTTA | TGCTTAATCAGTGAGG | |
| | (#71736) | TTTTC | CACC | |
| | | (SEQ ID NO: 13) | (SEQ ID NO: 14) | |
| phaZ | Cnambio 1 | Down_F3: | Down_R3: | 870 bp |
| flanking | DNA | GCATTGGTAAGCAAA | CGCCTGCAGAATCAGA | |
| downstream | | GTCGGCAGTGAGCCC | AGCAATACGGCGGAGG | |
| | | (SEQ ID NO: 15) | (SEQ ID NO: 16) | |

Deletion strain verification

| | | | | |
|---|---|---|---|---|
| phaZ | Cnambio 1 | ACACATACTCGCAAA | ATGAACAGTTGGCCGA | 767 bp |
| (RSGAB023 | DNA | ACCAACG | GGTGC | |
| 86) | | (SEQ ID NO: 17) | (SEQ ID NO: 18) | |
| Amp^{R} phaZ | Cnambio 1 | TTGTATAACACGCGG | CCGACTTTGCTTACCAA | 960 bp |
| delction | DNA, and phaZ | AACCCCTATTTGTTTA | TGCTTAATCAGTGAGG | |
| cassette) | deletion | TTTTC | CACC | |
| | cassette | (SEQ ID NO: 19) | (SEQ ID NO: 20) | |
| Kan^{R} | PG2K plasmid | TGAATGGACCAATAA | TGTCGTTCTGTCCACTC | 736 bp |
| | Addgene | TAATGACTAG | TTAATCC | |
| | (#71742) | (SEQ ID NO: 21) | (SEQ ID NO: 22) | |

Abbreviation: phaC: PHA synthase; phaZ: PHA depolymerase; citA: Citrate synthase; rpoD: RNA polymerase sigma factor; Kan^{R}: Kanamycin resistance gene; Amp^{R}: Ampicillin resistance gene; bp: Base pairs; kDa: Kilo Daltons; NA: Not applicable.

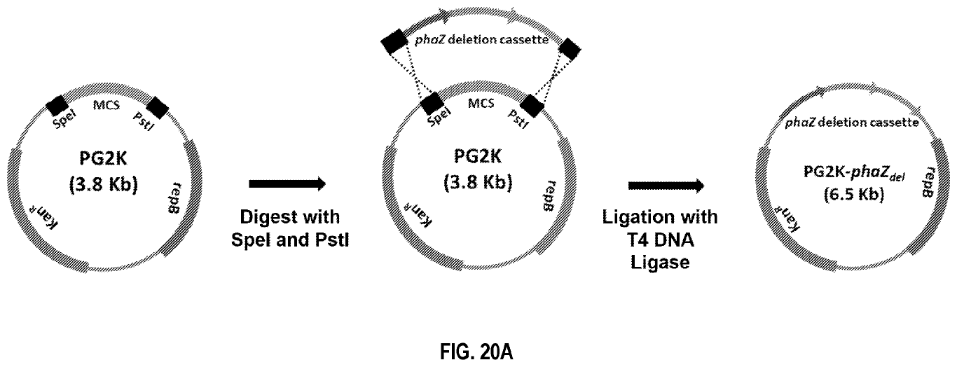

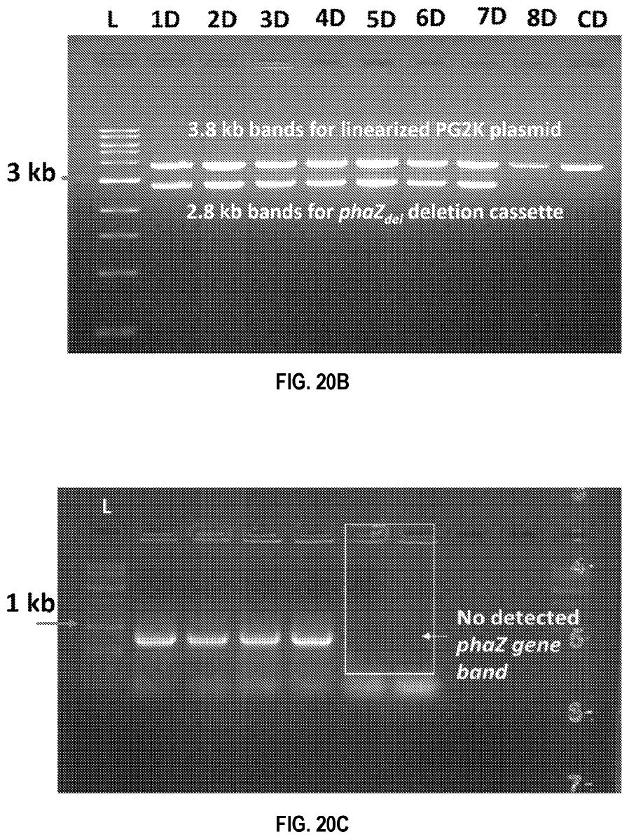

Following the PCR scheme illustrated in FIG. 18, a 2700 bp phaZ_{del} cassette was successfully generated (FIG. 19A-C). The resulting phaZ_{del} cassette was purified by electrophoresis, digested with correct restriction enzymes, ligated to PG2K plasmid (FIG. 20A) and transformed into E. coli DH5alpha, TOP 10, INV110, and BL21. The positive E. coli colonies containing the deletion construct were confirmed by selection on Kan 50 μg/mL, and Amp 100 μg/mL plates. The positive E. coli transformants were also confirmed by isolating the plasmids and digesting them with SpeI, and PstI. The appearance of two separate yet clear bands for linearized PG2K plasmid (3.8 Kb) and phaZ_{del} cassette (2.8 Kb) confirmed the presence of phaZ gene deletion cassette in the transformant PG2K plasmid (FIG. 20B).

Following this verification, the PG2K-phaZ gene cassette propagated in E. coli DH5alpha, TOP10, INV110, and BL21 were transformed into cnambio1. The cnambio1 accepted the plasmid from all the four E. coli strains, with no evident inconvenience. The results indicated that the cnambio1 RM systems did not affect pGK2K mobilization to a large extent. Following this transformation, few of the cnambio1 colonies were subjected to a series of steps to force the integration of Amp^{R} gene at the genomic allele of phaZ gene by double crossover HR, resulting in replacement of phaZ gene in the genome of cnambio1 with the Amp^{R} gene in deletion cassette. Briefly, gene deletion typically demands (a) initial integration of the PG2K-phaZ_{del} plasmid by a single cross-over event at the phaZ gene locus, aided by homologous phaZ upstream and downstream flanking regions, as well by the use of a temperature-sensitive repB replicon to force for the loss of the autonomously replicating PG2K plasmid, followed by (b) extensive screening of the mutants with selection for a second homologous cross-over event.

Out of approximately 1200 colonies screened, almost 99% of the colonies were found to maintain their wild type of genotype (i.e., still contained the phaZ gene in their genome but with the Kan^{R} phenotype). Nevertheless, two potential mutants that are phenotypically negative for growth on Kan plates, as well as negative for the presence of phaZ gene in the genome were identified (FIG. 20C).

Low percentage of knockouts is possibly due to the fact that PG2K vector used has a repB as a thermostable origin of replication that is indeed insensitive to temperature and can support the growth of transformants in presence of kanamycin even above 65° C. (at 68° C.). Ideally for a suicide vector it is essential that the plasmid should cease to replicate itself over 65° C. Any colonies growing above this temperature would thus have integrated the complete vector into the genome. Other Geobacillus suicide knock-out vectors such as PTM031, pSTE12, pUB110, pUB31 that have proven studies for gene knockouts can also be used to improve the frequency of knockout mutants.

The features disclosed in the foregoing description or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The inventions being thus described, it will be obvious that the same may be varied in many ways. Such variations are not to be regarded as a departure from the spirit and scope of the inventions and all such modifications are intended to be included within the scope of the following claims. The above specification provides a description of the manufacture and use of the disclosed compositions and methods. Since many embodiments can be made without departing from the spirit and scope of the invention, the invention resides in the claims.

What is claimed is:

1. A method for degrading lignocellulosic biomass, the method comprising:
adding a composition comprising Geobacillus thermodenitrificans strain cnambio1 (NRRL B-68082), or a mutant thereof, to the biomass to form a mixture; and incubating the mixture under conditions suitable for growth of the strain.

2. The method of claim 1, wherein the biomass is unprocessed.

3. The method of claim 1, wherein the biomass is pretreated.

4. The method of claim 1, wherein the lignocellulosic biomass is the sole carbon and energy source in the mixture.

5. The method of claim 1, wherein the biomass comprises wood, wood pulp, wood chips, sawdust, hardwood, softwood, newsprint, cardboard, paper pulp, corn fiber, corn grain, corn cobs, corn husks, corn stover, grasses, wheat, wheat straw, barley, barley straw, oat straw, oat hulls, hay, rice, rice straw, switchgrass, cord grass, rye grass, miscanthus, reed canary grass, waste paper, paper, fruit pulp, vegetable pulp, distillers grain, rice hulls, rice straw, cotton, hemp, flax, sisal, sugar cane bagasse, sugar cane straw, beet pulp, sorghum, soy, soybean stover, canola straw, flowers, or any mixtures thereof.

6. The method of claim 1, wherein the biomass comprises corn stover.

7. The method of claim 1, wherein the incubating is at a temperature of about 55° C. to about 60° C.

8. The method of claim 1, wherein the incubating is at a pH of about 7 to about 8.

9. The method of claim 1, wherein the incubating is carried out for a time of about 24 hours to about 10 days.

10. The method of claim 1, wherein the composition comprises Geobacillus thermodenitrificans strain cnambio1 (NRRL B-68082).

11. The method of claim 10, wherein the strain further comprises at least one genetic modification.

12. The method of claim 11, wherein the strain is genetically modified to: (a) recombinantly express an enzyme, optionally wherein the enzyme is a cellulase; (b) overexpress one or more genes of the phaCAB operon; or (c) knockout or knockdown expression of a Pha depolymerase (PhaZ).

13. A method for producing a polyhydroxyalkanoate (PHA) from biomass, the method comprising:
adding a composition comprising Geobacillus thermodenitrificans strain cnambio1 (NRRL B-68082), or a mutant thereof, to the biomass to form a mixture;
incubating the mixture under conditions suitable for growth of the strain to produce the PHA; and
extracting the PHA from the cells of the strain.

14. The method of claim 13, wherein the biomass is unprocessed.

15. The method of claim 13, wherein the biomass is pretreated.

16. The method of claim 13, wherein the biomass is the sole carbon and energy source in the mixture.

17. The method of claim 13, wherein the biomass comprises wood, wood pulp, wood chips, sawdust, hardwood, softwood, newsprint, cardboard, paper pulp, corn fiber, corn grain, corn cobs, corn husks, corn stover, grasses, wheat, wheat straw, barley, barley straw, oat straw, oat hulls, hay, rice, rice straw, switchgrass, cord grass, rye grass, miscanthus, reed canary grass, waste paper, paper, fruit pulp, vegetable pulp, distillers grain, rice hulls, rice straw, cotton, hemp, flax, sisal, sugar cane bagasse, sugar cane straw, beet pulp, sorghum, soy, soybean stover, canola straw, flowers, or any mixtures thereof.

18. The method of claim 13, wherein the PHA is a medium chain length PHA (mcl-PHA).

19. The method of claim 13, wherein the PHA has a number average molecular weight between about 10,000 and about 15,000 g/mol and a weight average molecular weight between about 20,000 and about 30,000 g/mol.

20. The method of claim 13, wherein the PHA is heat stable at a temperature of at least about 350° C.

21. The method of claim 13, wherein the extracting is with an organic solvent.

22. The method of claim 21, wherein the organic solvent comprises chloroform or propylene carbonate.

23. The method of claim 13, wherein the incubating is at a temperature of about 55° C. to about 60° C.

24. The method of claim 13, wherein the incubating is at a pH of about 7 to about 8.

25. The method of claim 13, wherein the incubating is carried out for a time of about 24 hours to about 10 days.

26. The method of claim 13, wherein the composition comprises Geobacillus thermodenitrificans strain cnambio1 (NRRL B-68082).

27. The method of claim 26, wherein the strain further comprises at least one genetic modification.

28. The method of claim 27, wherein the strain is genetically modified to: (a) recombinantly express an enzyme, optionally wherein the enzyme is a cellulase; (b) overexpress one or more genes of the phaCAB operon; or (c) knockout or knockdown expression of a Pha depolymerase (PhaZ).

29. A composition comprising Geobacillus thermodenitrificans strain cnambio1 (NRRL B-68082), or a mutant thereof.

30. The composition of claim 29, wherein the strain comprises a 16S ribosomal RNA sequence having at least about 95%, at least about 98%, or at least about 99% sequence identity with the sequence of SEQ ID NO: 1.

31. The composition of claim 29, further comprising a carrier.

32. The composition of claim 29, wherein the strain further comprises at least one genetic modification.

33. The composition of claim 32, wherein the strain is genetically modified to: (a) recombinantly express an enzyme, optionally wherein the enzyme is a cellulase; (b) overexpress one or more genes of the phaCAB operon; or (c) knockout or knockdown expression of a Pha depolymerase (PhaZ).

34. A polyhydroxyalkanoate (PHA) extracted from Geobacillus thermodenitrificans strain cnambio1 (NRRL B-68082) or a mutant thereof.

35. The PHA of claim 34, wherein the PHA is a medium chain length PHA (mcl-PHA).

36. The PHA of claim 34, wherein the PHA has a number average molecular weight between about 10,000 and about 15,000 g/mol and a weight average molecular weight between about 20,000 and about 30,000 g/mol.

37. The PHA of claim 34, wherein the PHA is heat stable at a temperature of at least about 350° C.

38. A method for transforming exogenous DNA into Geobacillus cells, the method comprising:
electroporating a suspension comprising the exogenous DNA and cells with (a) two or more square waveform pulses of about 0.75 kV to about 1.25 kV each, or (b) an exponential decay waveform pulse of at least about 2.5 kV.

39. The method of claim 38, wherein the electroporating comprises four square waveform pulses.

40. The method of claim 38, wherein each square waveform pulse has a duration of about 5 ms.

41. The method of claim 38, wherein the exponential decay waveform pulse has resistance of about 400Ω to about 800 Ω.

42. The method of claim 38, wherein the exponential decay waveform pulse has a capacitance of about 10 μF to about 50 μF.

43. The method of claim 38, wherein the cells are grown to an OD₆₀₀ of about 1.4 to about 1.8.

44. The method of claim 38, wherein the suspension comprises about 800 ng to about 1200 ng of the exogenous DNA.

45. The method of claim 38, wherein the exogenous DNA is linear or circular.

46. The method of claim 38, wherein the cells are Geobacillus thermodenitrificans strain cnambio1 (NRRL B-68082) cells.
